(19) United States
(12) Patent Application Publication
(10) Pub. No.: US 2013/0236429 A1
(43) Pub. Date: Sep. 12, 2013

(54) ENGINEERED VASCULAR ADIPOSE TISSUE

(71) Applicants: Juan M. Melero-Martin, Newton, MA (US); Joyce E. Bischoff, Weston, MA (US)

(72) Inventors: Juan M. Melero-Martin, Newton, MA (US); Joyce E. Bischoff, Weston, MA (US)

(73) Assignee: CHILDREN'S MEDICAL CENTER CORPORATION, Boston, MA (US)

(21) Appl. No.: 13/825,081

(22) PCT Filed: Sep. 22, 2011
(86) PCT No.: PCT/US11/52673

§ 371 Date: May 22, 2013

Related U.S. Application Data
(60) Provisional application No. 61/385,643, filed on Sep. 23, 2010.

(51) Int. Cl. A61K 35/36; A61K 35/28

(52) U.S. Cl. CPC A61K 35/36; A61K 35/28
USPC 424/93.7

(57) ABSTRACT

Embodiments of the invention relate to methods, compositions and kits for the in vivo formation of vascularized new adipose tissue in a subject. Combination of endothelial progenitor cells (EPCs) and mesenchymal progenitor cells (MPCs) implanted in vivo in a subject work synergistically to promote the formation vascularized new adipose tissue.

### CROSS REFERENCE TO RELATED APPLICATION

This application claims benefit under 35 U.S.C. §119(e) of the U.S. Provisional Application No. 61/385,643 filed on Sep. 23, 2010, the contents of which are incorporated herein by reference in its entirety.

### GOVERNMENT SUPPORT

This invention was made with Government support under Grant No.: K99 EB009096 awarded by the National Institutes of Health. The Government has certain rights in the invention.

### BACKGROUND OF INVENTION

The majority of the millions of plastic and reconstructive surgical procedures performed each year are to repair soft tissues that result from traumatic injury (e.g., significant burns), tumor resection (e.g., mastectomy and carcinoma removal) and congenital defects. These defects typically result from the loss of a large volume of adipose tissue. To date, there is no ideal filler material which is satisfactory in all cases. Additionally, the success of using autologous fat tissue grafts to repair soft tissue defects has been limited due to the lack of sufficient revascularization; 40-60% of the graft volume is typically lost due to insufficient blood supply. Therefore, there is large interest in developing strategies to engineer autologous volumes of adipose tissue that are useful in plastic, cosmetic and reconstructive surgical procedures.

### SUMMARY OF THE INVENTION

Embodiments of the present invention relate to in vivo neovascularization of newly created tissues and also relate to the in vivo formation of new tissue from the differentiation of progenitor cells implanted in a subject. In particular, embodiments of the present invention relate to vasculogenesis and adipogenesis in vivo.

Embodiments of the present invention are based on the development of new methodology for engineering in vivo vascularized adipose tissue that can be used in plastic, cosmetic and reconstructive surgical procedures. The inventors showed that autologous human blood-derived endothelial progenitor cells (EPCs) and bone marrow derived mesenchymal progenitor cells (bmMPCs) or adipose tissue derived mesenchymal progenitor cells (watMPCs), when implanted in vivo, (1) create a dense microvascular networks that allows blood perfusion through the implants, and (2) the progenitor MPCs differentiate into adipose tissue by mimicking the surrounded subcutaneous fat tissue present at the site of implantation. In some embodiments, surrounding progenitor cells are recruited to the site of implant and these recruited cells then differentiate to adipocytes. The result is a fully vascularized autologous adipose tissue. Such an engineered tissue is useful for plastic and reconstructive surgery. The inventers also show that implanting EPCs alone or MPCs alone do not result in a well vascularized adipose tissue for plastic, cosmetic and reconstructive purposes. Therefore, the problem of insufficient blood supply in newly formed tissue or a tissue graft is solved by the neovascularization of the newly formed tissue using a composition of EPCs and MPCs.

Accordingly, in one embodiment, provided herein is a composition for use in promoting vascularized adipose tissue formation comprising an enriched population of isolated endothelial progenitor cells (EPCs) and an enriched population of isolated mesenchymal progenitor cells (MPCs), wherein the EPCs and MPCs induce the formation of new blood vessels with functional connections to a host vasculature and the formation of new adipose tissue.

In one embodiment, provided here is a method of promoting in vivo neovascularization and vascularized adipose tissue formation in a subject in need thereof, the method comprises implanting into the subject a composition comprising an enriched population of isolated EPCs and an enriched population of isolated MPCs, wherein the EPCs and MPCs induce the formation of new blood vessels with functional connections to the subject's vasculature and the formation of new adipose tissue differentiated from the MPCs and/or recruited from the surrounding tissue of the implant site.

In one embodiment, the composition further comprises pre-differentiated MPCs and the pre-differentiated MPCs contribute to the formation of new adipose tissue at the implant site. In one embodiment, the MPCs have been pre-differentiated in vitro to adipocytes. In another embodiment, the MPCs are adipocytes isolated from adipose tissues. In one embodiment, the pre-differentiated MPCs are further expanded and cryopreserved.

In another embodiment, provided herein is a method of promoting in vivo neovascularization and vascularized adipose tissue formation in a subject in need thereof, the method comprises implanting into the subject a composition comprising an enriched population of isolated EPCs and an enriched population of pre-differentiated MPCs, wherein the EPCs induce the formation of new blood vessels with functional connections to the subject's vasculature and the pre-differentiated MPCs forms the new adipose tissue at the implant site.

The inventors demonstrated the formation of fat tissue using EPCs with either bone marrow (bmMSC) or fat (watMSC) MSCs. This indicated that the source of MSC does not change the overall result of the methods described herein for forming adipose tissues in vivo.

In some embodiments, the EPCs are derived from a source selected from a group consisting of bone marrow, cord blood, peripheral blood and blood vessel walls, and the MPCs are derived from a source selected from a group consisting of amniotic fluid, bone marrow, cord blood, peripheral blood and adipose tissue, e.g., white adipose tissue and brown adipose tissue. In some embodiments, the progenitor cells are both obtained from a sample of peripheral blood.

In one embodiment of the methods described herein, neovascularization is promoted initially followed by the induction or production of new adipocytes (fat cells) at the implant site.

In one embodiment, the progenitor cells are expanded in vitro after isolation from the various sources in order to increase the number of cells available for implant. In one embodiment, the expanded progenitor cells are cryopreserved prior to use. In other embodiments, the progenitor cells are cryopreserved first, thawed and expanded in vitro at a later time when needed. In other situations, the progenitor cells are cryopreserved first, thawed and expanded in vitro at a later time and expanded progenitor cells are then cryopreserved a second time prior to use. In all embodiments involving cryopreserved cells, progenitor and pre-differentiated cells, the cells are thawed prior to use.

In one embodiment, the progenitor cells are autologous to a recipient. In this way, the recipient will have new vascularized adipose tissue generated from his own progenitor cells.

In another embodiment, the progenitor cells are allogenic and HLA type matched to a recipient. Here, the new vascularized adipose tissues are not generated from the recipient's own progenitor cells but from a donor's progenitor cells that are allogenic and HLA type matched to the recipient.

The inventors also demonstrated in vivo adipose tissue formation using a collagen/fibrin/fibronectin mixture as the matrix or filler material together with the EPCs and MPCs. Other matrix or filler materials tested are a photo-crosslinkable gelatin methacrylate hydrogel (GelMA) and a mouse extracellular extract (MATRIGEL™) as the matrix.

In one embodiment, the composition of enriched populations of EPCs and MPCs are mixed with a filler material/matrix material/scaffold and the mixture containing both progenitor cells types are delivered simultaneously into the subject. In one embodiment, the filler material/matrix material/scaffold is a biocompatible material for use in vivo in a subject depending on the plastic, cosmetic, tissue engineering and reconstructive purposes. A biocompatible material is a synthetic or natural material used to replace part of a living system or to function in intimate contact with living tissue. In some embodiments, the filler material/matrix material/scaffold is a synthetic or natural material. In one embodiment, the filler material is an extracellular matrix, such as polymer scaffolds made of materials such as polyester based absorbable, hyaluronic acid, fibrin, silk, collagen, polyethylene glycol (PEG), and chemically modified alginate.

In one embodiment, the both types of progenitor cells are delivered simultaneously into the subject; both types of cell are mixed prior to deliver into the subject. In other embodiments, each type of progenitor cells is delivered sequentially to the subject. For example, EPCs are delivered first to the subject followed by MPCs and/or pre-differentiated MPCs.

In one embodiment, the delivery is by direct injection at the site of need. In one embodiment, the site of seed is an adipose tissue. In other embodiments, the site of seed is any tissue in the subject.

In one embodiment, the composition of progenitor cells is delivered, implanted or contacted by direct injection to an adipose tissue. In another embodiment, the composition of progenitor cells is delivered, implanted or contacted by direct injection to areas of the body that require new adipose tissue to provide shape and form for the organism, e.g., to fill in the void left by certain surgery or injury.

In some embodiments, the adipose tissue or the areas that are in need of new adipose tissue to give shape and form are found in areas selected from a group consisting of the skin, head, face, torso, muscle, legs, and arms. In other embodiments, the areas that are in need of new adipose tissue to give shape and form can be any part of an organism's body.

In one embodiment, the enriched population of EPCs is at least 10% but not more than 90% of the total cells in the composition.

In one embodiment, the enriched population of MPCs is at least 10% but not more than 90% of the total cells in the composition.

In one embodiment, the enriched population of EPCs is 40% of the total cells in the composition.

In one embodiment, the pre-differentiated MPCs are at least 10% and not more that 90% of the composition.

In one embodiment, the MPCs and the pre-differentiated MPCs, if pre-differentiated MPCs are used, collectively make up at least 40% of the total cells in the composition.

In one embodiment, the composition comprises about 40% EPCs and about 60% the MPCs.

Another embodiment of the present invention provides for a composition for promoting in vivo neovascularization and vascularized adipose tissue formation comprising: (a) an enriched population of isolated EPCs; (b) an enriched population of isolated MPCs; (c) an enriched population of pre-differentiated MPCs; and (d) a pharmaceutically acceptable carrier.

In one embodiment, the EPCs of the composition comprise at least 10% but not more than 90% of the total cells in the composition.

In one embodiment, the MPCs of the composition comprise at least 10% but not more than 90% of the total cells in the composition.

In one embodiment, the pre-differentiated MPCs are at least 10% and not more that 90% of the composition.

In one embodiment, the MPCs and the pre-differentiated MPCs collectively make up at least 40% of the total cells in the composition.

In one embodiment, the composition further comprises an extracellular matrix/filler material/scaffold material, such as polymer scaffolds made of materials such as polyester based absorbable, fibrin, silk hyaluronic acid, collagen, polyethylene glycol (PEG), HYLAFORM® and CAPTIQUE™, RESTYLANE™, RADIESSE™ (hydroxylapatite), SCULPTRA™, poly-L-Lactic Acid (PLLA), PURAMATRIX™, photo-crosslinkable gelatin methacrylate hydrogels, polymethylmethacrylate beads (PMMA microspheres) and chemically modified alginate. In one embodiment, the composition further comprises at least one extracellular matrix/filler material/scaffold material.

In another embodiment, provided herein is a kit comprising: (a) an enriched population of isolated EPCs; (b) an enriched population of isolated MPCs; and (c) a progenitor differentiation factor for pre-differentiating the MPCs.

In one embodiment, the kit further comprises at least one extracellular matrix, filler material, or a biocompatible scaffold, such as polymer scaffolds made of materials such as polyester based absorbable, hyaluronic acid, fibrin, silk, collagen, polyethylene glycol (PEG), photo-crosslinkable gelatin methacrylate hydrogels, hydroxylapatite, PURAMATRIX™, HYLAFORM® and CAPTIQUE™ and chemically modified alginate.

In one embodiment, the extracellular matrix filler material, or a biocompatible scaffold of the kit or composition has a stiffness property of about 1 Pa to 600 Pa.

In one embodiment, the pre-differentiated MPCs are differentiated in vitro.

In one embodiment, the pre-differentiated MPCs are differentiated to pre-adipocytes or adipocytes.

In one embodiment, the kit further comprises an enriched population of pre-differentiated MPCs.

In another embodiment, the kit further comprises instructions on the use of the cells in the kit for promoting in vivo neovascularization and vascularized adipose tissue formation in a tissue.

In one embodiment, provided herein in a method of promoting vascularized adipose tissue formation in a subject in need thereof comprising implanting a tissue with a composition comprising the EPCs and MPCs described herein or using the kit comprising EPCs, MPCs and pre-differentiated MPCs described herein.

In one embodiment of the methods of promoting vascularized adipose tissue formation further comprises selecting the subject for implanting the composition.

### BRIEF DESCRIPTION OF THE DRAWINGS

FIGS. 1A-1B show an exemplary working protocol for in vivo vasculogenesis and adipogenesis.

FIG. 1A shows an exemplary working protocol for in vivo vasculogenesis.

FIG. 1B shows an exemplary working protocol for in vivo adipogenesis following vasculogenesis.

FIG. 2A shows an exemplary working protocol for the isolation and characterization of fat-derived MPCs (watMSCs) from mice.

FIG. 2B shows exemplary cell sorting data of the cell surface markers that are present and/or absent in the isolated watMSCs from mice.

FIG. 2C is a summary of the cell surface markers characterized in the isolated watMSCs from mice.

FIG. 2D shows that the isolated watMSCs from mice are multipotent and can be differentiated into the adipogenic, chondrogenic or osteogenic lineages.

FIGS. 3A-3B show an exemplary working protocol for the isolation of watMPCs and the in vivo implantation experiments in mice.

FIG. 3A shows an exemplary working protocol for the isolation of watMSCs from mice.

FIG. 3B shows an exemplary working protocol for the in vivo implantation of isolated watMSCs in athymic (nu/nu) nude mice.

FIG. 4A is a stained section of the implant showing engineered vasculature. The triangles indicate patent blood vessels.

FIG. 4B is a histogram showing that significantly more microvessels are formed after one week in vivo in the implants having the composition with combined cells of cbEPCs and watMSCs.

FIGS. 5A-5D show significant adipogenesis after 4 weeks in implants comprising a composition comprising mouse watMSC (watMSC) and human code blood EPCs (cb-EPCs) in MATRIGEL™.

FIG. 5A shows the engineered adipose tissue formed at 4 week after implantation.

FIG. 5B is a histogram showing that significantly more adipocytes (by % fraction) are present after four weeks in vivo in the implants having the composition with combined cells of cbEPCs and watMSCs compared to implants having the composition with only watMSCs.

FIG. 5C is a histogram showing that there are significantly more adipocytes per unit area of the implant after four weeks in vivo for the implants having the composition with combined cells of cbEPCs and watMSCs compared to implants having the composition with only watMSCs.

FIG. 5D is a histogram showing that the sizes of the adipocytes present in the implants after four weeks in vivo are approximately the same for the implants having the composition with combined cells of cbEPCs and watMSCs and the implants having the composition with only watMSCs.

FIG. 6A shows the mesenchymal marker CD90, the endothelial marker CD31, and hematopoietic marker CD45 in h-bmMSC, h-watMSC, and human cord-blood derived endothelial colony-forming cells (h-cbECFC) via flow cytometry analysis. Dotted lines represent cells stained with fluorescent antibodies. Isotype-matched controls are overlaid in a solid line on each panel.

FIG. 6B shows the typical spindle morphology characteristic of MSC in culture. The morphology was the same irregardless of the source of the MSCs. Scale bars, 100 μm.

FIG. 6C shows the multilineage differentiation of human MSCs: 1) adipocytes (adipogenesis); 2) osteocytes (osteogenesis); and 3) chondrocytes (chondrogenesis).

FIG. 7A diagrams the strategy for the 7-day implantation and extraction experiment of using a composition of hMSC and hECFC. After extraction, the hECFC, hMSC, mouse endothelial cells (mEC) and the mouse stromal cell populations were identified and quantified in n=4 replicates.

FIG. 7B diagrams the histograms showing the distribution of hECFC (mCD45−, hCD31+) in the implant after 7 days.

FIG. 7C diagrams the histograms showing the distribution of hMSC (mCD45−, hCD31−, hCD90+) in the implant after 7 days.

FIG. 7D diagrams the histograms showing the distribution of mouse endothelial cells, mEC (mCD45−, hCD31−, hCD90−, mCD31+) in the implant after 7 days.

FIG. 7E diagrams the histograms showing the distribution of mouse stromal cells (mCD45−, hCD31−, hCD90−, mCD31−, mCD29+) in the implant after 7 days.

FIG. 8A shows the general experimental protocol for implanting a composition comprising h-watMSCs, h-ECFC and a matrix/scaffold material on the back of 6-week-old GFP-expressing SCID mouse by subcutaneous injection. Implants were harvested after 7 days. Macroscopic view of an explant at day 7 is depicted.

FIG. 8B shows that the H&E staining of sections taken from the explants at day 7 revealed the presence of numerous blood vessels containing murine erythrocytes.

FIG. 8C shows the extent of microvessel densities of implants and quantified by counting lumenal structures containing erythrocytes. Data are shown as histograms. Bars represent the mean microvessel density determined from four replicate implants (one mouse each) ±S.D.

FIGS. 9A-9D show significant adipogenesis after 4 weeks in implants comprising a composition comprising human watMSC (h-watMSC) and human-ECFC (h-ECFC) in MATRIGEL™.

FIG. 9A are representative H&E staining of sections taken from the explants at 4 weeks depicted at 10× and 40× magnifications, demonstrating the presence of adipose tissue (adipocytes) formed with human watMSCs.

FIG. 9B shows the quantified data of adipocyte area fraction (%) in the H&E pictures. All groups and compared to native adipose tissue. Bars represent mean±S.D determined from four replicate implants (one mouse each).

FIG. 9C shows the quantified data of adipocyte density (adipocyte/mm²) in the H&E pictures. All groups and compared to native adipose tissue. Bars represent mean±S.D determined from four replicate implants (one mouse each).

FIG. 9D shows the quantified data of average adipocyte size (um²) were in the H&E pictures. All groups and compared to native adipose tissue. Bars represent mean±S.D determined from four replicate implants (one mouse each).

FIGS. 10A-10D show significant adipogenesis after 4 weeks in implants comprising a composition comprising human watMSC (h-watMSC) and human-ECFC (h-ECFC) in a collagen/fibrin-based gel.

FIG. 10A are representative H&E staining of sections taken from the explants at 4 weeks depicted at 10× and 40× magnifications, demonstrating the presence of adipose tissue (adipocytes) formed with human watMSCs. A macroscopic view of explant at 4 weeks are depicted in insets.

FIG. 10B shows the quantified data of adipocyte area fraction (%) in the H&E pictures. All groups and compared to native adipose tissue. Bars represent mean±S.D determined from four replicate implants (one mouse each).

FIG. 10C shows the quantified data of adipocyte density (adipocyte/mm²) in the H&E pictures. All groups and compared to native adipose tissue. Bars represent mean±S.D determined from four replicate implants (one mouse each).

FIG. 10D shows the quantified data of average adipocyte size (um²) were in the H&E pictures. All groups and compared to native adipose tissue. Bars represent mean±S.D determined from four replicate implants (one mouse each).

FIG. 11 shows significant adipogenesis after 4 weeks in implants comprising a composition comprising human watMSC (h-watMSC) and human-ECFC (h-ECFC) in 1M GelMA. Representative H&E stained sections of one implant after 4 weeks in vivo is shown. A macroscopic view shown in the inset.

### DETAILED DESCRIPTION OF THE INVENTION

The present invention relates to methods of vasculogenesis and adipogenesis in vivo.

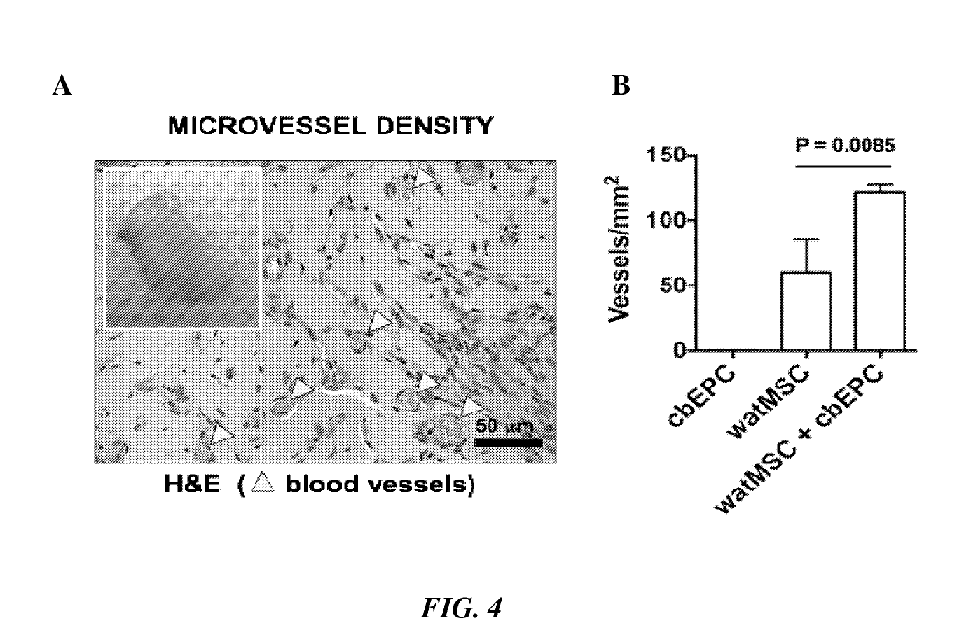

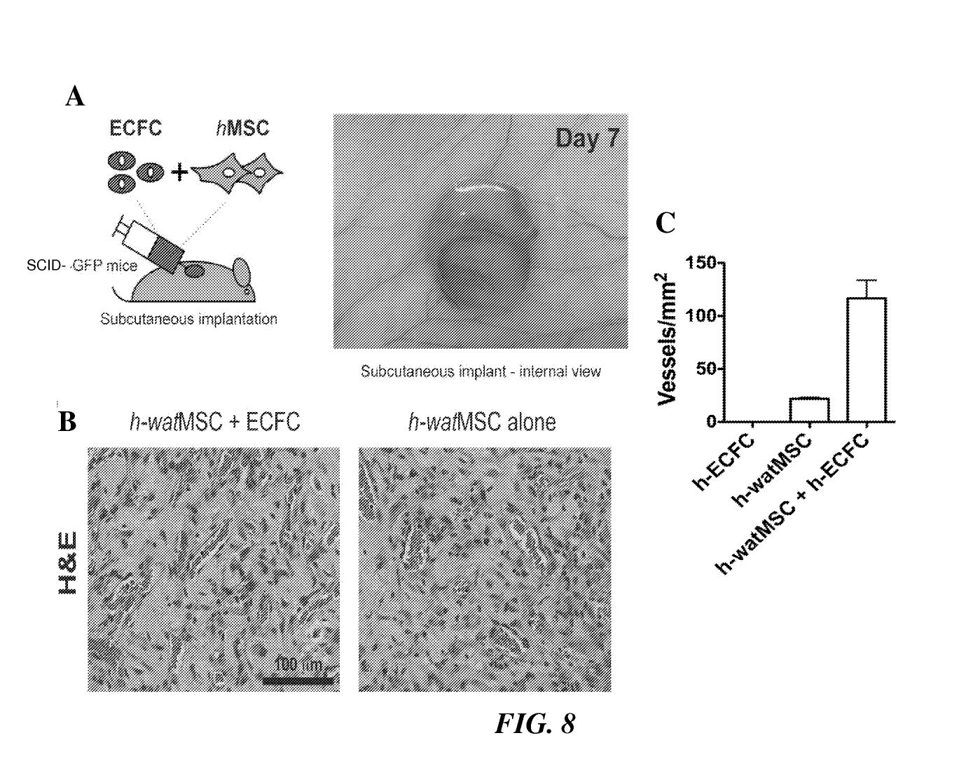

Embodiments described herein are based on a dual progenitor cell-based method for vasculogenesis and adipogenesis in vivo. The inventors showed that implanting human blood-derived endothelial progenitor cells (EPCs) in combination of white adipose tissue (wat)-derived mesenchymal progenitor cells (watMPCs) produces functional and long-lasting vascular networks in newly formed adipose tissue in vivo. The EPCs alone do not form or produce any functional blood vessels (see FIGS. 4 and 8C) and the MPCs alone produce low density of functional blood vessels. In contrast, the combination of EPCs and MPCs dramatically increased the functional blood vessels (see FIGS. 4 and 8C).

This EPC/MPC dual progenitor cell-based methods enable vascularization of newly create adipose tissue with an adequate vascular beds. When EPCs and MPCs are implanted subcutaneously, they first create a functional vascular bed, after which they proceed to transform into adipose tissue mimicking the surrounding adipose tissue present at the site of implantation.

Accordingly, in one embodiment, provided herein is a method of promoting in vivo neovascularization and vascularized adipose tissue formation in a subject in need thereof comprising implanting into a tissue of a subject with a composition comprising an enriched population of isolated EPCs and an enriched population of isolated MPCs, wherein the EPCs and MPCs induce the formation of new blood vessels with functional connections to the subject's vasculature and the formation of new adipose tissues.

In one embodiment, provided herein is a composition comprising an enriched population of isolated endothelial progenitor cells (EPCs) and an enriched population of isolated mesenchymal progenitor cells (MPCs) for use of promoting vascularized adipose tissue formation, wherein the EPCs and MPCs induce the formation of new blood vessels with functional connections to a host vasculature and the formation of new adipose tissue.

In another embodiment, provided herein is a composition for use in promoting in vivo neovascularization and vascularized adipose tissue formation, the composition comprises: (a) an enriched population of isolated EPCs; (b) an enriched population of isolated MPCs and; (d) a pharmaceutically acceptable carrier.

In one embodiment, the composition further comprises an enriched population of pre-differentiated MPCs. Other non-exclusive components that can be included in the composition include filler/matrix/scaffold materials, growth factors, anti-infection agents (e.g., antibiotics, anti-fungal etc), anti-inflammatory agents and other cell types that are not EPCs or MPCs. These non-EPCs or non-MPCs can be easily distinguished from EPCs or MPCs respectively by their cell surface markers by any methods known in the art, e.g., fluorescence activated cell sorting (FACS).

Another embodiment of the present invention provides for a composition for promoting in vivo neovascularization and vascularized adipose tissue formation comprising: (a) an enriched population of isolated EPCs; (b) an enriched population of isolated MPCs; (c) an enriched population of pre-differentiated MPCs and; (d) a pharmaceutically acceptable carrier.

In another embodiment, provided herein is a kit comprising: (a) an enriched population of isolated EPCs; (b) an enriched population of isolated MPCs; and (c) a progenitor differentiation factor for pre-differentiating the MPCs in the kit.

In one embodiment, the invention provides a kit comprising a composition comprising: (a) an enriched population of isolated EPCs; (b) an enriched population of isolated MPCs; (c) an enriched population of pre-differentiated MPCs and; (d) a pharmaceutically acceptable carrier.

In another embodiment, the kit further comprises instructions on the use of the cells in the kit for promoting in vivo neovascularization and vascularized adipose tissue formation in a subject in need thereof. Other components that can be included in the kit include but are not limited to filler/matrix/scaffold materials, growth factors, anti-infection agents (e.g., antibiotics etc), other cell types that are not EPCs or MPCs, sterile syringes, sterile gloves, sterile vesicles and sterile containers. In one embodiment, the kit comprises at least one filler/matrix/scaffold material. In some embodiment, the kit comprises several filler/matrix/scaffold materials, so as to provide the user a choice of filler/matrix/scaffold material to be used depending on the plastic, cosmetic, tissue engineering or reconstructive purpose.

In one embodiment, the kit further comprises instructions on the use of the cells in the kit for promoting in vivo neovascularization and vascularized adipose tissue formation in a tissue in need thereof.

In one embodiment, provided herein in a method of promoting vascularized adipose tissue formation in a subject in need thereof comprising implanting a tissue with a composition comprising EPCs and MPCs described herein or using a kit comprising EPCs, MPCs, differentiation factors and/or pre-differentiated MPCs described herein.

In one embodiment of the methods for promoting vascularized adipose tissue formation further comprises selecting the subject for implanting the composition.

In one embodiment, the composition consists essentially of an enriched population of isolated EPCs and an enriched population of isolated MPCs. In another embodiment, the composition consists essentially of an enriched population of isolated EPCs, an enriched population of isolated MPCs and an enriched population of pre-differentiated MPCs.

In one embodiment, the composition consists of an enriched population of isolated EPCs and an enriched population of isolated MPCs. In another embodiment, the composition consists of an enriched population of isolated EPCs, an enriched population of isolated MPCs and an enriched population of pre-differentiated MPCs.

In one embodiment, the kit consists essentially of an enriched population of isolated EPCs, an enriched population of isolated MPCs and a progenitor differentiation factor for pre-differentiating the MPCs in the kit. In another embodiment, the kit consists essentially of an enriched population of isolated EPCs, an enriched population of isolated MPCs, a progenitor differentiation factor for pre-differentiating the MPCs in the kit and an enriched population of pre-differentiated MPCs. In yet another embodiment, the kit consists essentially of an enriched population of isolated EPCs, an enriched population of isolated MPCs, and an enriched population of pre-differentiated MPCs.

In one embodiment, the kit consists of an enriched population of isolated EPCs, an enriched population of isolated MPCs and a progenitor differentiation factor for pre-differentiating the MPCs in the kit. In another embodiment, the kit consists of an enriched population of isolated EPCs, an enriched population of isolated MPCs, a progenitor differentiation factor for pre-differentiating the MPCs in the kit and an enriched population of pre-differentiated MPCs. In yet another embodiment, the kit consists of an enriched population of isolated EPCs, an enriched population of isolated MPCs, and an enriched population of pre-differentiated MPCs.

These embodiments are useful for providing vascularized soft tissue replacement in the body of an organism or subject in need thereof, for example, from traumatic injury or disease (e.g., flesh eating bacteria infection and HIV infection), tumor resection, and congenital defects.

The majority of the millions of plastic, cosmetic and reconstructive surgical procedures performed each year are to repair soft tissue that result from traumatic injury (e.g., significant burns), tumor resection (e.g., mastectomy and carcinoma removal), and congenital defects (e.g., vascular anomalies such as infantile hemangiomas). These defects typically result from the loss of a large volume of subcutaneous adipose tissue (Brey, et al., 2000, IEEE Eng Med Biol Mag 19:122-125; Katz, A. J., et al., 1999, Clin Plast Surg 26:587-603, viii; Patrick, C. W., Jr. 2001, Anat Rec 263:361-366). Plastic surgeons are constantly burdened with the clinical needs for tissue augmentations and/or reconstructions to improve the aesthetic contour of soft tissues (Alster, T. S., and West, T. B. 2000, Plast Reconstr Surg 105:2515-2525; discussion 2526-2518; Beahm, E. K., et al., 2003, Clin Plast Surg 30:547-558, viii; Patrick, C. W., Jr., et al., 1999, Tissue Eng 5:139-151); the current clinical options are either autologous adipose tissue grafts (obtained through liposuction) or synthetic implants. Autologous soft-tissue grafts are commonly used because allografts, xenografts, and synthetic materials have complications such as pathogen transmission and immune rejection issues (Butler, D. L., and Awad, H. A. 1999, Clin Orthop Relat Res:S324-332; Eppley, B. L. 1999, Plast Reconstr Surg 104:1761-1783; quiz 1784-1765; Hart, D. 1999, Plast Surg Nurs 19:137-142, 147). Various levels of clinical success have been reported with the use of autologous soft tissue (Butler, D. L., and Awad, H. A. 1999, Clin Orthop Relat Res:S324-332; Eppley, B. L. 1999, Plast Reconstr Surg 104:1761-1783; quiz 1784-1765; Erol, O. O., and Spira, M. 1990, Plast Reconstr Surg 86:510-518; Kononas, T. C., et al., 1993, Plast Reconstr Surg 91:763-768; Zuk, P. A., et al., Tissue Eng 7:211-228). However, this success has been limited due to the lack of sufficient revascularization (40-60% of the graft volume is typically lost) (C. W., Jr. 2001, Anat Rec 263:361-366, Patrick, C. W., Jr. 2000, Semin Surg Oncol 19:302-311). The fat grafts never acquire sufficient vascularity, so centralized graft blood flow is not adequate for long-term survival of the tissue, and often leads to tissue resorption (Patrick, C. W. 2004, Annu Rev Biomed Eng 6:109-130). As for other alternatives, there is no ideal filler material that satisfies all clinical needs. Synthetic materials such as silicone or saline implants have the advantage of endless supply and have been documented to replace missing soft tissues with various levels of clinical success. However, synthetic materials have drawbacks, such as rupture, leakage, dislocation, and suboptimal biocompatibility (Stosich, M. S., and Mao, J. J. 2007, Plast Reconstr Surg 119:71-83). Non-synthetic or biocompatible materials present the problem of short longevity: they start being degraded once implanted in vivo and therefore they constitute non-permanent solutions. These materials include bioengineered hyaluronic acid derivatives, which are known to provide safe and effective soft-tissue augmentation in the comprehensive approach to nonsurgical facial rejuvenation (Alam, M., and Dover, J. S. 2007, In Plast Reconstr Surg.; Arlette, J. P., and Trotter, M. J. 2008, In Dermatologic Surgery; Lambros, V. S. 2007, In Plast Reconstr Surg.; Narins, R. S., et al., 2007, In Dermatologic Surgery). Current hyaluronic acid fillers do not require pre-injection skin testing and produce reproducible, longer-lasting (though non-permanent) results compared with other fillers, such as collagen (Rohrich, R. J., et al., 2007, In Plast Reconstr Surg.), and are therefore among the preferred fillers recommended by clinicians. However, despite the fact that hyaluronic acid-based fillers have increased tissue longevity compared to fat and collagen, the effects are also not permanent and on average patients return to baseline at approximately 3-9 months (Rohrich, R. J., et al., 2007, In Plast Reconstr Surg.). As a result, repeat treatments are usually necessary to maintain the corrections. In fact, patients are counseled to anticipate supplemental implantations to achieve and maintain optimal correction.

For plastic and reconstructive surgeries to be successful, adequate vascularization is of upmost importance. Often times, adequate vascularization requires neovascularization, i.e. de novo synthesis of new blood vessels. The creation of vascular networks is crucial for the success of therapeutic neovascularization in regenerative medicine such as tissue-engineered (TE) organs and tissues used in plastic and reconstructive surgery. To guarantee an appropriate provision of nutrients, gas exchange, and elimination of waste products, engineered tissues must have the capacity to generate a vascular network that anastomoses with the host vasculature shortly after implantation. Increased blood flow via new vascular network can speed development, differentiation, recovery and healing in damages and transplanted tissues. Currently, there are no TE constructs clinically available with an inherent microvascular bed, and therefore successes in TE have been restricted to the replacement of relatively thin (skin) or avascular tissues (cartilage), where post-implantation neovascularization from the host is sufficient.

Tissues in need of neovascularization include all TE constructs and implanted tissues that are greater than 2 mm in thickness and these tissues are normally vascularized in the human body. For example, tissue engineered cardiac muscles, bladder, pancreas, and liver to name a few. The neovascularization of such engineered tissues, when implanted into a mammal, ensures the survival and functionality of the tissue in the mammalian host. In accordance with the invention disclosed herein, the presence of EPCs and MPCs in the TE construct enables the tissue to form de novo blood vessels that anastamose with the existing host circulatory network at the site of implantation. Formation of an adequate vascular network will provide a constant supply of oxygen and nutrients for the engineered tissue as well as facilitate efficient removal of toxic metabolic waste products. A constant supply of oxygen and nutrients is necessary for the engineered tissue to grow, remodel, and perform its biological function in the body.

To overcome this problem of neovascularization, several therapeutic strategies have been proposed and tested. These strategies center on promoting angiogenesis—in-growth of microvessels by delivering angiogenic molecules such as VEGF, either as proteins or via gene transfer to the tissues needing neovascularization or re-neovascularization. However, these strategies cannot provide rapid and complete neovascularization of thick tissues, engineered or natural. A complete neovascularization of tissues, whether the tissues are engineered tissue or naturally existing tissue in an organism, requires the additional process of vasculogenesis.

In vivo vasculogenesis can be promoted by exploiting the inherent vasculogenic ability of endothelial cells (ECs). Earlier studies using human umbilical vein ECs (HUVECs) and human microvascular ECs (HDMECs) showed the feasibility of engineering microvascular networks in vivo (Koike, N., et. al., 2004, Nature, 428:138-9; Nor, J. E., et. al., 2001, Lab. Invest. 81:453-63; Schechner, J. S. et. al., 2000, Proc. Natl. Acad. Sci. USA, 97: 9191-6). However, the clinical use of mature ECs derived from autologous vascular tissue is limited by the difficulty of obtaining sufficient quantities of cells with minimal donor site morbidity. In addition, the studies by Schechner and Nor required genetic modification of the mature EC using the anti-apoptotic gene bcl-2, which could participate in alteration of the cells to a cancerous state.

Embodiments of the methods, compositions and kit relate to using at least two types of progenitor cells, EPC and MPC, for the neovascularization and adipogenesis within at least one filler/matrix/scaffold material in plastic, cosmetic, tissue regeneration and reconstructive surgery. Populations of these progenitor cells are isolated from sources such as circulating peripheral blood, umbilical cord blood, bone marrow, and adipose tissue. The isolated population of progenitor cells is then enriched by various methods known in the art and expanded through multiple cell divisions to produce sufficient number of progenitor cells for the methods of the invention disclosed herein.

During the process of neovascularization, both EPCs and MPCs work together to form de novo blood vessels. New branched of blood vessels form from existing blood vessels, and they join up with the de novo vessels to form a network. The EPCs mature and differentiate into ECs which forms the tunica intima—thinnest and inner walls of the blood vessels; the MPCs give rise to smooth muscle cells that make up the bulk of the tunica media—the thickest layer and tunica adventitia—connective tissue layer of a blood vessel.

There are several advantages to using a combination of EPCs and MPCs for vascular network formation. Progenitor cells are immature or undifferentiated cells, and they have greater cell division capability. Therefore, it is possible to culture in vitro the desired progenitor cells to obtain sufficient quantities for the neovascularization of engineered tissues and in therapeutic vasculogenesis. Moreover both EPCs and MPCs are present in the circulating blood and can be isolated from a single sample of blood, for example, circulating peripheral blood. The isolated EPCs and MPCs can then be expanded in vitro prior to use. Furthermore, the MPCs can be differentiated in vitro into adipocytes.

The methods, compositions and kits described herein provide solutions to the inadequate vascularization problem. In addition, and also the progenitor cells furnish the source of the major filler materials that fill in the space voided by tissue loss due injury, aging, surgery, disease etc. Since the filler material used for implantation comprises progenitor cells proliferate to supply the progeny cells that would differentiate to living adipose cells, with proper vascularization and under normal in vivo conditions, these cells remain viable for a longer period of time compared to the synthetic filler materials. As a result, the subject would not have to have supplemental implantations to achieve and maintain optimal correction or repair.

In the example, the inventors showed that functional microvascular beds formed inside the implants after one week of implantation. This was shown by the presence of an extensive network of human blood vessels containing erythrocytes, indicating rapid formation of functional anastomoses with the host mouse vasculature. In this vascular network, the implanted EPCs were restricted to the luminal aspect of the vessels, while MPCs are adjacent to lumens, confirming their role as perivascular cells. Importantly, the engineered vascular networks remain patent after four weeks in vivo. The vessels are functional because the vessel is patent, i.e., the lumen of the vessel is open and clear of obstruction, and was filled with erythrocytes (red blood cells).

In the example, the inventors also showed that over time, the cells within the MATRIGEL™ implants undergo a process of in vivo remodeling characterized by stabilization of total cellularity and redistribution of perivascular cells. Alpha-SMA-expressing cells are initially detected (day 7) around the lumenal structures and throughout the MATRIGEL™ implants. However, over time the expression of α-SMA is progressively restricted to perivascular locations, as expected in normal stabilized vasculature. Finally, after 21-28 days in vivo, the presence of adipocytes is identified by staining with an anti-perilipin antibody, indicating a process of integration between the implants and the surrounding mouse adipose tissue.

Furthermore, the inventors showed that implanting a combination of watMPCs/cbEPCs gave significantly larger fraction of adipose tissue than implanting just watMPCs alone. The combination of watMPCs/cbEPCs gave a beneficial effect of high initial microvascular density for later adipogenesis. Accordingly, in some embodiments of the methods, composition and kit described herein, a combination of MPCs and EPCs are used. In some embodiments of the methods, composition and kit described herein, MPCs alone or EPCs alone are not used.

In some embodiments, the combination of MPCs and EPCs are used in conjunction with other bioactive agents such as growth factors etc. as described else in the application. The bioactive agents promote cell proliferation and differentiation of the implanted EPCs and MPCs. The bioactive agents can be added with the EPCs and MPCs during implantation or added after EPCs and MPCs implantation. One skilled in the art would be able to determined with minimal experimentation the ideal approach when using bioactive agents with EPCs and MPCs.

In some embodiments, anti-infection agents are included in combination of MPCs and EPCs to prevent infection by pathogens, e.g., bacteria, fungi etc, during the formation of vascularized adipose tissue. Any anti-infection agents are known in the art can be used, e.g., antibiotics and anti-fungals. A skill physician will be able to select an appropriate anti-infection agent for the particular subject and the particular vasculogenic/adipogenic purposes

In some embodiments, anti-inflammation agents are included in combination of MPCs and EPCs to prevent an inflammation reaction by the subject that can result in the rejection of the progenitor cells and/or the tissue formed therefrom. Any anti-inflammation agents are known in the art can be used, e.g., corticosteroids. A skill physician will be able to select an appropriate anti-inflammation agent for the particular subject and the particular vasculogenic/adipogenic purposes

It is envisioned that after longer periods of time in vivo, the EPC/MPC dual cell-based implants described herein will be predominantly comprise of adipose tissue with a functional supporting vascular bed, and that they will integrate with the surrounding tissue at the site of implantation. The functional supporting vascular bed will provide adequate blood flow to the newly formed adipose tissue. This will significantly improve the performance of current strategies that uses filler materials without cells or supporting vasculature.

In some embodiments of the methods, composition and kits described herein, the EPCs are derived from a source selected from a group consisting of bone marrow, cord blood, peripheral blood and blood vessel walls, and the MPCs are derived from a source selected from a group consisting of amniotic fluid, bone marrow, cord blood, peripheral blood and adipose tissue. In some embodiments, the progenitor cells are both obtained from a sample of peripheral blood.

In one embodiment of the methods, composition and kits described herein, the progenitor cells are autologous to a recipient. The donor and the recipient of the EPCs and MPCs are the same patient. This greatly reduces the problem of tissue rejection of engineered adipose tissues or immune response rejecting the progenitor cells that are implanted into the body of the recipients, e.g., in the reconstruction of their organs, tissues and wounds. Examples of organs and tissues for which embodiments of the invention disclosed herein are applicable include but are not limited to the heart, muscles, skin, adipose tissue, brain, bone, liver, lungs, intestines, legs, limbs, and kidneys. Basically, embodiments of the invention can be used to restore the shape and form of a patient in need thereof. In this way, the recipient will have new vascularized adipose tissue generated from his own progenitor cells.

In one embodiment, the autologous EPCs and MPCs can be used for the invention disclosed herein. Prior to major surgery to repair certain defects, a patient can donate a sample of human bone marrow, peripheral blood and/or adipose tissue for the isolation and expansion of EPCs and MPCs. In another embodiment, EPCs and MPCs can be isolated for the purpose of pre-banking the progenitor cells in high risk populations, for example those serving in the military. In the event that a solder is injured and results in missing a part of or a whole organ, tissue, and/or body parts such as facial bones, the solder's previously banked EPCs and MPCs can be utilized for TE projects to reconstruct the missing organ, tissue, and/or body parts. Enriched populations of EPCs and MPCs are obtained from suitable sources disclosed herein. A composition comprising of an enriched population of isolated autologous EPCs and an enriched population of isolated autologous MPCs can used for implanting in the patient. In another aspect, the composition can be injected directly to the area of the body or the tissue/organs that need filling and repair.

In another embodiment, the EPC and/or MPC progenitor cells are allogenic and HLA type matched to a recipient. Here, the new vascularized adipose tissues are not generated from the recepient's own progenitor cells but from the donor's progenitor cells that are allogenic and HLA type matched to the recipient.

In one embodiment, the EPCs and MPCs are human leukocyte antigen (HLA) typed matched for the recipient of the cells. In one embodiment, EPCs and MPCs are isolated and expanded from a single donor and the progenitor cells are matched for at least 4 out of 6 alleles of the HLA class I: HLA-A and HLA-B; and HLA class II: DRB1 with the recipient. In another embodiment, EPCs and MPCs are isolated and expanded from different donors and the progenitor cells are HLA type matched for at least 4 out of 6 alleles of the HLA class I: HLA-A and HLA-B; and HLA class II: DRB1 with the recipient.

In one embodiment, provided herein is a bank of cells which comprises a composition comprising an enriched population of isolated EPCs and an enriched population of isolated MPCs. In one embodiment, the bank of cells further comprises an enriched population of pre-differentiated MPCs. In one embodiment, the bank of cells comprises a composition comprising an enriched population of isolated EPCs. In another embodiment, the bank of cells comprises a composition comprising an enriched population of isolated MPCs. In one embodiment, the bank of cells comprises a composition comprising an enriched population of pre-differentiated MPCs. In one embodiment, the progenitor cells are isolated and enriched in vitro and then cryopreserved as banks of cells. In one embodiment, the progenitor cells are isolated and expanded in vitro prior to cryopreservation for the bank of cells. The progenitor cells can be isolated and enriched by any method known in the art, including those described in the EPC and MPC sections respectively. The progenitor cells can also be cryopreserved by any method known in the art, including those described in the section on cryopreservation. The MPCs can also be pre-differentiated with specific differentiating factor before cryopreservation as banks of cells. The MPCs can be differentiated in vitro by any method known in the art, including those described in the section on MPCs. When EPCs and MPCs are needed for any neovascularization and formation of new adipose tissues, the cryopreserved EPCs and MPCs of the cell bank can be thawed and utilized. In other embodiments, the cryopreserved EPCs and MPCs of the cell bank can also be thawed and then utilized, further expanded to increase the numbers, and/or pre-differentiated before used in implantation into patients in need thereof.

In one embodiment, the recipient of a composition comprising an enriched population of isolated EPCs and an enriched population of isolated MPCs is a mammal. Examples of mammals include but are not limited to dog, cat, sheep, goat, monkeys, pigs and human. In a preferred embodiment, the recipient is a human. As used herein, the terms “recipient”, “subject”, “patient”, “organism” and “host” are used interchangeably.

In one embodiment, the methods described herein comprise selecting a subject for implanting. The subject can be some missing tissue due to infection by “flesh-eating” bacteria, breast masectomy or burn injuries.

In one embodiment, the composition of enriched populations of EPCs and MPCs are mixed with a filler material and the both types of progenitor cells are delivered simultaneously.

In one embodiment, the filler material is an extracellular matrix, such as polymer scaffolds made of materials such as polyester based absorbable, hyaluronic acid, fibrin, silk, collagen, polyethylene glycol (PEG), photo-crosslinkable gelatin methacrylate hydrogels, hydroxylapatite, chemically modified alginate, and HYLAFORM® and CAPTIQUE™ from GENZYME.

In one embodiment, the filler material, the extracellular matrix or scaffold material has a stiffness property of about 1 Pa to 600 Pa.

In one embodiment, the composition of progenitor cells is delivered, implanted or contacted by direct injection to an adipose tissue. In another embodiment, the composition of progenitor cells is delivered, implanted or contacted by direct injection to areas of the body that require new adipose tissue to provide shape and form for the organism.

In some embodiments of the methods, compositions and kits described herein, the tissue or the areas that are in need of new adipose tissue to give shape and form is found in areas selected from a group consisting of the skin, head, face, torso, muscle, legs, and arms. In other embodiments, the areas that are in need of new adipose tissue to give shape and form can be any part of a subject's body.

In one embodiment of the methods, compositions and kits described herein, the enriched population of EPCs is at least 10% but not more than 90% of the total cells in the composition.

In one embodiment of the methods, compositions and kits described herein, the enriched population of MPCs is at least 10% but not more than 90% of the total cells in the composition.

In one embodiment of all aspects of the methods, compositions and kits described herein, the enriched population of EPCs is 40% of the total cells in the composition. In another embodiment of all aspects of the methods, compositions and kits described herein, the enriched population of EPCs comprises 40% of the total cells in the composition.

In one embodiment of all aspects of the compositions described herein further comprises pre-differentiated MPCs. In one embodiment, the MPCs have been pre-differentiated in vitro. In one embodiment, the MPCs have been pre-differentiated to pre-adipocytes or adipocytes.

In one embodiment, the pre-differentiated MPCs are at least 10% and not more than 90% of the total cells in the composition or kit.

In one embodiment, the MPCs and the pre-differentiated MPCs collectively comprises at least 40% of the total cells in the composition or kit.

In one embodiment, the composition comprises about 40% EPCs and about 60% the MPCs.

In one embodiment of all aspect of the compositions or kits described herein further comprises an extracellular matrix, such as polymer scaffolds made of materials such as polyester based absorbable, hyaluronic acid, collagen, polyethylene glycol (PEG), silk, fibrin, photo-crosslinkable gelatin methacrylate hydrogels, chemically modified alginate, PURAMATRIX™, HYLAFORM® and CAPTIQUE™.

In one embodiment, the kit further comprises at least one extracellular matrix or a biocompatible scaffold, such as polymer scaffolds made of materials such as polyester based absorbable, hyaluronic acid, collagen, polyethylene glycol (PEG), and chemically modified alginate.

In another embodiment, the kit further comprises instructions on the use of the cells in the kit for promoting in vivo neovascularization and vascularized adipose tissue formation in a tissue in need thereof.

In some embodiments of all aspects of the methods, compositions and kits described herein, the promotion of neovascularization and/or vascularized adipose tissue formation occurs in vitro, in vivo or ex vivo depending on the purpose e.g., in a tissue regeneration or tissue engineering situation, the adipose tissue can be formed ex vivo or in vitro.

### DEFINITIONS

As used herein, the term “angiogenesis” refers to the formation of new blood vessels from pre-existing blood vessels. The term “angiogenesis” and “vasculogenesis” are used interchangeably. Blood vessel formation occurring by a de novo process where EPCs and MPCs migrate, assemble and differentiate in response to local cues (such as growth factors and extracellular matrices) to form new blood vessels.

As used herein, “neovascularization” refers to the formation of functional vascular networks that are perfused by blood or blood components. Neovascularization includes angiogenesis, budding angiogenesis, intussuceptive angiogenesis, sprouting angiogenesis, therapeutic angiogenesis and vasculogenesis.

As used herein, the term “adipogenesis” refers to the formation of new adipose tissue. The new adipose tissue can be constituted from progenitor cells that differentiate into an adipogenic lineage to give rise to adipocytes. Alternatively, the new adipose tissue can be constituted from MPCs that have been pre-differentiated to pre-adipocytes, adipocytes or a combination of both. The progenitor cells can be cells that were implanted to a location or that were recruited in vivo to the site of implant or a specific location.

As used herein, the term “adjacent” or “surrounding” with regard to a location, e.g., a wound or site of implant of the composition described herein, refers to being close enough to that location for molecules such as growth factors to spread by passive diffusion from the adjacent tissue to that location, and for cells injected at the adjacent tissue to migrate to that location.

As used herein, the term “progenitor” cell refers to an immature or undifferentiated cell, typically found in post-natal animals. Progenitor cells can be unipotent or multipotent. As used herein, progenitor cells refers to either EPCs or MPCs, or both EPCs and MPCs. The EPCs are also known ECFCs. The MPCs are also known as MSCs.

As used herein, the term “autologous” refers to a situation in which the donor of progenitor cells is also the recipient of the progenitor cells.

As used herein, “allogenic cells” refer to cells that are from one or more different donors and the donor(s) is not the same person as the recipient of the donated progenitor cells. Therefore, the “allogenic cells” are not genetically identical to the recipient's cells.

The term “isolated” as used herein signifies that the cells are placed into conditions other than their natural environment. The term “isolated” does not preclude the later use of these cells thereafter in combinations or mixtures with other cells.

As used herein, the term “enriched” with respect to a population of cells means that at least 90% of the cells in the population are the same, i.e., of the same cell type. For example, an enriched population of MPCs would have at least 90% of the cells that are PDGFR-β positive and CD45 negative. Enrichment of cells can be achieved by any methods known in the art or those described in the section on EPCs and MPCs, e.g., by density gradient centrifugation and fluorescent activated cell sorting (FACS).

As used herein, the term “expanding” refers to increasing the number of like cells through cell division (mitosis). The term “proliferating” and “expanding” are used interchangeably.

As used herein, “cryopreservation” refers to the preservation of cells by cooling to low sub-zero temperatures, such as (typically) 77° K or −196° C. (the boiling point of liquid nitrogen). Cryopreservation also refers to storing the cells at a temperature between 0-10° C. in the absence of any cryopreservative agents. At these low temperatures, any biological activity, including the biochemical reactions that would lead to cell death, is effectively stopped. Cryoprotective agents are often used at sub-zero temperatures to preserve the cells from damaged due to freezing at low temperatures or warming to room temperature.

As used herein, “composition” refers to an injectate, substance or a combination of substances which can be delivered into a tissue, an organ, or a tissue engineered construct such a gel-like extracellular matrix or a biocompatible scaffold. Exemplary compositions include, but are not limited to, a suspension of progenitor cells in a suitable physiologic carrier such as saline.

As used here, “delivery” refers to providing a composition to a treatment site in an injured tissue through any method appropriate to deliver the functional composition to the treatment site; or deliver to a TE construct such as a biocompatible scaffold. Non-limiting examples of delivery methods include direct injection at the treatment site, percutaneous delivery for injection, and other delivery methods well known to persons of ordinary skill in the art.

As used herein, the terms “tissue regeneration”, “tissue engineering” and “regenerative medicine” are related terms and used interchangeably.

As used herein, the word “repair”, means the natural replacement of worn, torn or broken components with newly synthesized components. The word “healing”, as used herein, means the returning of torn and broken organs and tissues (wounds) to wholeness.

As used herein, the term “tissue engineered construct” or TE construct” or construct refers to a product made by assembling adherent cells on to a scaffold using the techniques of tissue engineering that is known in the art.

As used herein, the term “biocompatible” refers to the ability to replace part of a living system or to function in intimate contact with living tissue. A biocompatible material is a synthetic or natural material used to replace part of a living system or to function in intimate contact with living tissue. Biocompatible materials are intended to interface with biological systems to evaluate, treat, augment or replace any tissue, organ or function of the body.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic composition is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

As used herein, the terms “administer” refers to the placement of the composition of EPCs/MPCs and/or pre-determined MPCs by any appropriate route which results in an effective tissue repair and/or tissue reconstruction tissue in the subject.

As used herein, the term “comprising” or “comprises” is used in reference to compositions, methods, kits and respective component(s) thereof, that are essential to the invention, yet open to the inclusion of unspecified elements, whether essential or not. The use of “comprising” indicates inclusion rather than limitation.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

### Endothelial Progenitor Cells (EPC)

EPCs are primitive cells that originate in the bone marrow or derived from the blood vessel walls. EPCs are released into the bloodstream. These circulating, bone marrow-derived EPCs go to areas of blood vessel injury to help repair the damage. They have the ability to expand and differentiate into ECs, the cells that make up the inner lining of blood vessels, and are known to participate in both vasculogenesis and vascular homeostasis.

EPCs are also known as endothelial colony forming cells (ECFCs). Therefore, as used herein, “EPCs” and “ECFCs” are used interchangeably.

As used herein, the term “h-ECFC” refers to human endothelial colony forming cells.

As used herein, the term “cbEPCs” refers to human umbilical cord blood endothelial progenitor cells.

Sources of EPCs include human umbilical cord blood, human bone marrow, human circulating peripheral blood, and blood vessel walls. In one embodiment, EPCs of the invention can be isolated from circulating peripheral blood and the umbilical cord blood. From a sample of blood, the mononuclear cell fraction (MNC) of the blood is obtained by percoll gradient centrifugation. This MNC fraction can be further purified for EPCs based on the CD34/CD133+ surface markers of EPCs and then expanded in culture using EPC medium. EPC medium: EGM-2 (Endothelial Basal Medium (EBM-2)+SingleQuots; hydrocortisoneis excluded; Lonza, Walkersville, Md.), 20% fetal bovine serum (FBS) and 1× glutamine-penicillin-streptomycin (GPS; INVITROGEN™, Carlsbad, Calif.). In one embodiment, human serum, either autologous or allogeneic AB serum, or human platelet rich plasma supplemented with heparin (2 U/ml) can be used instead of FBS. Alternatively, the MNC fraction can be grown in tissue culture directly. Non-adherent cells are removed 48 hours later (for cord blood) and 4 days later (for periphery blood). After being in culture for 2-3 weeks, the cells are confluent and are then selected for CD31, another surface marker of EPCs. At this time the EPCs have a cobblestone-like morphology in culture, positive for the following markers: CD34, KDR, CD146, CD31, CD105, VE-cadherin, vWF, and eNOS; and negative for CD90, CD45, and CD14. In addition the EPCs response to the TNF-α by up regulating expression of E-selectin, ICAM-1 and VCAM-1. Over the course of the next 1-7 weeks in culture, the EPCs expand exponential with 30-70 cells population doublings. The EPCs and EC specific markers can be monitored by any methods known in the art, for example, flow cytometry using specific antibodies against the various cell surface markers.

In one embodiment, the enriched population of isolated EPCs is at least 90% positive for CD31 and VE-cadherin, and no more than 5% positive for at least one of the markers: CD90, CD45 and CD14.

In one embodiment, the isolated EPCs are singly or collectively negative for mesenchymal cell markers. In one embodiment, the isolated EPCs are singly or collectively negative for PDGFR-β. In another embodiment, the isolated EPCs are singly or collectively negative for CD 90 which is also known as Thy-1. In one embodiment, the population of isolated EPCs is no more than 5% positive for mesenchymal cell markers such as PDGFR-13 and/or CD 90.

In one embodiment, the isolated EPCs are singly or collectively negative for hematopoietic stem cell markers such as CD45 and CD11b. In one embodiment, the population of the isolated EPCs is no more than 5% positive for hematopoietic stem cell markers such as CD45 and CD11b.

Other methods of isolating, culture and expansion of EPCs are described by Jonathan M. Hill, 2003, NEJM, 348:593-600; Eggermann J, 2003, Cardiovasc Res., 58(2):478-86; Hristov, et al., 2003, Trends in Cardiovascular Medicine 13 (5): 201-6; Amelia Casamassimi et. al, 2007, J. Biochemistry, 141:503-11; and U.S. Pat. No. 5,980,887, and U.S. Patent application Nos. 2003/0194802, 2006/0035290, and 2006/010385 and are hereby incorporated by reference.

### Mesenchymal Progenitor Cells (MPC)

MPCs are cells derived from the mesoderm and they have a large capacity for self-renewal while maintaining their multipotency. MPCs are undifferentiated mesenchymal cells that are capable of expanding and differentiating into more than one specific type of mesenchymal tissue cells. Cell types that MPCs have been shown to differentiate into in vitro or in vivo include osteoblasts, chondrocytes, myocytes, and adipocytes. MPCs are also referred to as mesenchymal stem cells (MSC) and they are used herein interchangeably.

Sources of MPCs include human amniotic fluid, human bone marrow, human umbilical cord blood, human circulating peripheral blood, and human white adipose tissue. MPCs are isolated, for example, from the mononuclear cell fraction of umbilical cord blood or peripheral blood. The MNC fraction is grown in MPC culture media: EGM-2 (Endothelial Basal Medium (EBM-2)+SingleQuots; VEGF, bFGF, hydrocortisone, heparin are excluded; Lonza, Walkersville, Md.), 20% fetal bovine serum (FBS) and 1×GPS (Invitrogen, Carlsbad, Calif.). In one embodiment, human serum, either autologous or allogeneic AB serum, or human platelet rich plasma supplemented with heparin (2 U/ml) can be used instead of FBS. At this stage these MPCs have a mesenchymal-like morphology (spindle-like) and express specific mesenchymal cell markers (positive for CD90, α-SMA, Calponin, CD44, CD105, CD29 and CD146) and do not express hematopoietic (negative for CD14 and CD45) and endothelial cell markers (negative for CD31, VE-Cadherin and vWF) (Pitting et. al., 1999, Science 284:143-147; Kaviani et. al., 2001, J. Pediatr. Surg. 36: 1662-5; Kunisaki et al., 2007, J. Pediatr. Surg. 42:974-9).

White adipose tissue-derived mesenchymal stem cells (watMSCs) were isolated by digesting human subcutaneous fat pads in a solution containing 1 mg/mL collagenase A and 2.5 U/mL dispase at 37° C. for 1 hour. After filtered through a 100-1 μm cell strainer, cell suspension was plated on uncoated tissue culture plates using MSCGM medium (Lonza, Walkersville, Md.) supplemented with 10% FBS, 1×GPS and 10 ng/mL bFGF (R&D, Minneapolis, Minn.). Unbound cells were removed at 48 hours, and the bound cell fraction maintained in culture until 70% confluence using MSCGM medium. h-watMSC were subcultured on uncoated tissue culture plates using MSCGM medium.

Other methods of isolation and expansion of MPCs are described in Current Protocols in Stem Cell Biology (Mick Bhatia, et. al., ed., 2007, John Wiley and Sons, Inc.) and in U.S. Pat. No. 5,486,359, 6,387,367, 7,060,494 and these are hereby incorporated by reference in their entirety.

As used herein, the term “MPC” and “MSC” (mesenchymal stem cells) are used interchangeably.

As used herein, the term “bmMPC” refers to bone marrow-derived mesenchymal progenitor cells and the term “bmMSC” refers to bone marrow-derived mesenchymal stem cells.

As used herein, the term “bmMPC” and “bmMSC” are used interchangeably.

As used herein, the term “watMPS” refers to white adipose tissue-derived mesenchymal progenitor cells and the term “watMSC” refers to white adipose tissue-derived mesenchymal stem cells.

As used herein, the term “watMPC” and “watMSC” are used interchangeably.

In one embodiment, the enriched population of isolated MPCs is at least 90% positive for CD90 and no more than 5% positive for CD45 and/or CD31.

In one embodiment, the enriched population of isolated MPCs is positive for PDGFR-β and negative for CD45.

In one embodiment, the enriched population of isolated MPCs is at least 90% positive for PDGFR-13 and no more than 5% positive for CD45 and/or CD31.

In one embodiment, the isolated MPCs are negative for endothelial cell markers such as CD31, VE-Cadherin and/or CD34.

In one embodiment, the enriched population of isolated MPCs is no more than 5% positive for endothelial cell markers such as CD31, VE-Cadherin and/or CD34.

In one embodiment, the isolated MPCs are singly or collectively negative for hematopoietic stem cell markers such as CD45 and CD11b. In one embodiment, the enriched population of isolated MPCs is no more than 5% positive for hematopoietic stem cell markers such as CD45 and CD11b.

In one embodiment, the isolated EPCs and MPCs are autologous to a recipient.

In another embodiment, a single sample of peripheral blood can be used for isolating and expanding the EPCs and MPCs. After isolation and expansion, the EPCs and MPCs can be cryopreserved by any methods known in the art. In one embodiment, the isolated EPCs and MPCs can be cryopreserved by any methods known in the art or those described herein.

In one embodiment, the isolated MPCs are differentiated in vitro to adipocytes. The isolated MPCs used can be the isolated MPCs that have been expanded, or those that have been previously cryopreserved. Methods of differentiating MPCS to adipocytes are well known in the art. For example, using the Mesenchymal Stem Cell Adipogenesis Kit from Millipore cat. No. SCR020 which contains reagents that readily differentiate mesenchymal stem cells to an adipogenic lineage. The factors known to differentiate mesenchymal stem cells to adipocytes include but are not limited to dexamethasone, IBMX, insulin and indomethacin. An adipogenic lineage is assessed with Oil Red O staining of lipid vacuoles in mature adipocytes. Along with Oil Red O staining solution, a hematoxylin solution is provided in the kit to counterstain the cell nucleus.

In one embodiment, the following adipogenesis assay is used to differentiate isolated MPCs in vitro. Confluent MPCs are cultured for 10 days in DMEM low-glucose medium with 10% FBS, 1×GPS, and adipogenic supplements (5 μg/mL insulin, 1 μM dexamethasone, 0.5 mM isobutylmethylxanthine, 60 μM indomethacin). Differentiated adipocytes are assessed by Oil Red O staining.

In one embodiment, the adipocytes obtained from the differentiated MPCs in vitro constitute the pre-differentiated MPCs.

In another embodiment, the adipocytes are isolated from the adipose tissues from a subject and these then constitute the pre-differentiated MPCs. In one embodiment, the isolated adipocytes are expanded in vitro, by any method known in the art, prior to use in vasculogenesis and adipogenesis in a subject. Additionally, the isolated adipocytes can be cryopreserved prior to and/or after expansion in vitro. In some embodiments, these cells also constitute the pre-differentiated MPCs. Pre-differentiated MPCs stain positively for perilipin-A and are easily distinguished from undifferentiated MPCs.

### Cryopreservation of Cells

In one embodiment, the invention provides a cryopreserved composition comprising an enriched population of isolated EPCs and an enriched population of isolated MPCs; an amount of cryopreservative sufficient for the cryopreservation of the isolated progenitor cells; and a pharmaceutically acceptable carrier. In one embodiment, the cryopreserved composition comprises a composition comprising an enriched population of isolated EPCs; an amount of cryopreservative sufficient for the cryopreservation of the isolated EPCs; and a pharmaceutically acceptable carrier. In another embodiment, the cryopreserved composition comprises a composition comprising an enriched population of isolated MPCs; an amount of cryopreservative sufficient for the cryopreservation of the isolated MPCs; and a pharmaceutically acceptable carrier.

The EPCs and MPCs described can be cryopreserved by any method known in the art. Similarly, the pre-differentiated MPCs described can be cryopreserved by any method known in the art. For example, the cryopreservation procedure described in Current Protocols in Stem Cell Biology, 2007, (Mick Bhatia, et. al., ed., John Wiley and Sons, Inc.) which is hereby incorporated by reference it its entirety. Mainly when the EPCs or MPCs on a 10-cm tissue culture plate have reached at least 50% confluency, preferably 70% confluency, the media within the plate is aspirated and the progenitor cells are rinsed with phosphate buffered saline. The adherent progenitor cells are then detached by 3 ml of 0.025% trypsin/0.04% EDTA treatment. The trypsin/EDTA is neutralized by 7 ml of media and the detached progenitor cells are collected by centrifugation at 200×g for 2 min. The supernatant is aspirated off and the pellet of progenitor cells is resuspended in 1.5 ml of media. The harvested progenitor cells are cryopreserved at a density of at least 3×10³ cells/ml. An aliquot of 1 ml of 100% DMSO is added to the suspension of progenitor cells and gently mixed. Then 1 ml aliquot of this suspension of progenitor cells in DMSO is dispensed into cyrules in preparation for cryopreservation. The sterilized storage cryules preferably have their caps threaded inside, allowing easy handling without contamination. Suitable racking systems are commercially available and can be used for cataloguing, storage, and retrieval of individual specimens.

Other methods of cryopreservation of viable cells, or modifications thereof, are available and envisioned for use (e.g., cold metal-mirror techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, 4,559,298 and are incorporated hereby reference.

Recovering Progenitor Cells from the Frozen State

When the progenitor cells are needed for vasculogenesis and adipogenesis, such as when a tissue is being engineered for a patient or when a void left behind by surgery or injury needs filing, the frozen EPCs and MPCs can be thawed according to any methods known in the art such as those described herein, mixed in appropriate ratios and incorporated into the engineered tissue or the void of any tissue or organ as needed for reconstruction.

Frozen progenitor cells are preferably thawed quickly (e.g., in a water bath maintained at 37°-41° C.) and chilled on ice immediately upon thawing. In particular, the cryogenic vial containing the frozen progenitor cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In a particular embodiment, the thawing procedure after cryopreservation as described in Current Protocols in Stem Cell Biology 2007 (Mick Bhatia, et. al., ed., John Wiley and Sons, Inc.) is used. Immediately after removing the cryogenic vial from the cryo-freezer, the vial is rolled between the hands for 10 to 30 sec until the outside of the vial is frost free. The vial is then held upright in a 37° C. water-bath until the contents are visibly thawed. The vial is immersed in 95% ethanol or sprayed with 70% ethanol to kill microorganisms from the water-bath and air dry in a sterile hood. The contents of the vial are then transferred to a 10-cm sterile culture containing 9 ml of media using sterile techniques. The progenitor cells can then be cultured and further expanded in an incubator at 37° C. with 5% humidified CO₂.

It may be desirable to treat the progenitor cells in order to prevent cellular clumping upon thawing. To prevent clumping, various procedures can be used, including but not limited to, the addition before and/or after freezing of DNase (Spitzer, G., et al., 1980, Cancer 45:3075-3085), low molecular weight dextran and citrate, hydroxyethyl starch (Stiff, P. J., et al., 1983, Cryobiology 20:17-24).

The cryoprotective agent, if toxic in humans, should be removed prior to therapeutic use of the thawed progenitor cells. In embodiments employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet the cells, removal of the supernatant, and resuspension of the cells. For example, the intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

Other procedures which can be used, relating to processing of the thawed cells, include enrichment for adherent progenitor cells and expansion by in vitro culture as described supra.

In a preferred, but not required, embadiment, thawed cells are tested by standard assays of viability (e.g., trypan blue exclusion) and of microbial sterility as described herein, and tested to confirm and/or determine their identity relative to the recipient.

Endotoxin levels can be determined by the gel-clot limulus amebocyte lysate (LAL) test method in compliance with the US Food and Drug Administration's GMP regulations, 21 CFR §211. Acceptable endotoxin level is 5.0 EU/ml.

An aliquot of the cells will be taken prior to cryopreservation for mycoplasma PCR testing. The Mycoplasma PCR testing will be performed at a GMP approved facility using MycoSensor™ QPCR Assay Kit (Manufactured by Stratagene).

Methods for identity testing which can be used include but are not limited to HLA typing (Bodmer, W., 1973, in Manual of Tissue Typing Techniques, Ray, J. G., et al., eds., DHEW Publication No. (NIH) 74-545, pp. 24-27), and DNA fingerprinting, which can be used to establish the genetic identity of the cells. DNA fingerprinting (Jeffreys, A. J., et al., 1985, Nature 314:67-73) exploits the extensive restriction fragment length polymorphism associated with hypervariable minisatellite regions of human DNA, to enable identification of the origin of a DNA sample, specific to each individual (Jeffreys, A. J., et al., 1985, Nature 316:76; Gill, P., et al., 1985, Nature 318:577; Vassart, G., et al., 1987, Science 235:683), and is thus preferred for use.

### Formation of Functional Anastomoses

Neovascularization can be created in vivo using EPCs, MPCs and/or pre-differentiated MPC isolated and purified from umbilical blood cord, periphery blood, bone marrow or white adipose tissue. In the Example 1, implanted MATRIGEL™ xenographs containing 2:3 ratio of cbEPCs to watMPCs exhibited the presence of murine red blood cells-containing blood vessels seven days post-implantation. This indicated the formation of functional anastomoses with the murine circulatory system of the host. Therefore, microvascular networks can be created within a tissue using human autologous EPCs and MPCs obtained from umbilical cord blood, periphery blood, bone marrow or white adipose tissue. This invention can be applied widely to any area in a host body or to newly made tissues that require a blood supply, and even to any tissue in the body that is ischemic as a result of illness and diseases such as congestive heart failure, poor circulation, obesity, lymphatic obstructions and diabetes.

In one embodiment, the EPCs and MPCs are mixed together to achieve microneovascularization in vivo. In one embodiment, the mixing of EPCs and MPCs can be in vitro, e.g., before implanting into a subject. In another embodiment, the mixing of EPCs and MPCs is achieved in vivo, at the location of implantation, e.g., EPCs and MPCs are separately implanted to approximately the same location and the mixing occurs at the location after implanting. Just EPCs alone or just MPCs alone do not promote robust microneovascularization in vivo in the absence of the other cell type. In one embodiment, the cell composition comprising EPC and MPC comprises at least 10% of each cell type. In some embodiments, the percentage of EPC in the composition is about 10%, 20%, 30%, 40%, 50%, 60%, 70%, 80%, 90%, and all the percentages between 10-90%. In some embodiments, the percentage of MPCs in the composition is about 10%, 20%, 30%, 40%, 50%, 60%, 70%, 80%, 90%, and all the percentages between 10-90%. In one embodiment, the EPCs and MPCs are mixed to obtain a final 100% of total cells. In a preferred embodiment, the percentage ratio of EPC to MPC is 40%:60%. In other embodiments, the EPCs and MPCs are mixed to obtain a final 70-100% of total cells.

In one embodiment, the composition comprising EPCs and MPCs further comprises pre-differentiated MPCs. In one embodiment, the EPCs, MPCs and pre-differentiated MPCs are mixed together to achieve microneovascularization in vivo. In one embodiment, the mixing of EPCs, MPCs and pre-differentiated MPCs can be in vitro. In some embodiments, the percentage of pre-differentiated MPCs in the composition is about 10%, 20%, 30%, 40%, 50%, 60%, 70%, 80%, 90%, and all the percentages between 10-90%. In one embodiment, the EPCs, MPCs, and pre-differentiated MPCs are mixed to obtain a final 100% of total cells. In a one embodiment, the percentage ratio of EPC:MPC:pre-differentiated MPC is 40%:50%:10%. In other embodiments, the EPCs, MPCs and pre-differentiated MPCs are mixed to obtain a final 70%-100% of total cells of the composition. When the combined EPCs and MPCs make up less that 100% of the total cells in the composition, the remaining percentage of cells of the composition are not EPCs and not MPCs. These cells can include but are not limited other cell types such as EPCs and/or other MPCs differentiated progenies (e.g., osteocytes) and smooth muscle cells.

In one embodiment, the EPCs are capable of differentiating into ECs and forming small blood vessel in the presence of smooth muscle cells in vivo. For example, a 4:1 ratio mixture of EPCs and human saphenous vein smooth muscle cells (HSVSMC) in extracellular matrix material MATRIGEL was injected subcutaneously into mice and after a week in vivo, the implant contained numerous small blood vessels which tested positive for specific endothelial cell markers such as CD31 and α-smooth muscle actin (α-SMA).

In another embodiment, human saphenous vein smooth muscle cells (HSVSMC), human brain vascular smooth muscle cells (HBVSMC) Cat. #1100; human esophageal smooth muscle Cells (HESMC) Cat. #2710; human intestinal smooth muscle cells (HISMC) Cat. #2910; human colonic smooth muscle cells (HCSMC) Cat. #2940; human pulmonary artery smooth muscle cells (HPASMC) Cat. #3110; human bronchial smooth muscle cells (HBSMC) Cat. #3400; human tracheal smooth muscle cells (HTSMC) Cat. #3410; human bladder smooth muscle cells (HBdSMC) Cat. #4310; human aortic smooth muscle cells (HASMC) Cat. #6110; human umbilical vein smooth muscle cells (HUVSMC) Cat. #8020; human umbilical artery smooth muscle cells (HUASMC) Cat. #8030 can be used to achieve microneovascularization in vivo according to the method described. These cells are commercially available at ScienCell™ Research Laboratories.

### Formation of Functional Adipose Tissues

New adipose tissue can be created in vivo using the composition comprising EPCs, MPCs and/or pre-differentiated MPC isolated and purified from umbilical blood cord, periphery blood, bone marrow or white adipose tissue. In the Examples 1 and 2, implanted MATRIGEL™ xenographs containing 2:3 (40%:60%) ratio of cbEPCs to watMPCs exhibited the presence of adipocytes which stain positively for perilipin-A at four weeks post-implantation. The perilipin-A positive adipocytes were derived from the implanted MPCs, pre-differentiated MPCs or from MPCs recruited from the adjacent tissue of the implant site.

### Therapeutic Uses

An embodiment of the invention disclosed herein is a method of promoting neovascularization in newly formed adipose tissue and promoting vascularized adipose tissue formation in a subject in need thereof. The method comprises implanting in the subject a composition comprising an enriched population of isolated EPCs and an enriched population of isolated MPCs, wherein the EPCs and MPCs induce the formation of new blood vessels with functional connections to the subject's vasculature and the formation of new adipose tissue. The new adipose tissue can be derived from the differentiation of the implanted MPCs. Alternately, the new adipose tissue can be derived from the differentiation of MPCs that were recruited from the adjacent tissue of the implant site. It is foreseeable that the body uses a combination of implanted MPCs and recruited MPCs to form the new adipose tissue. It is also foreseeable that other types of cell are recruited to the implant site for making the new adipose tissue. Examples of other types of cell are progenitor and precursor cells and fibroblasts. In another embodiment, method comprises implanting in the subject a composition comprising an enriched population of isolated EPCs, an enriched population of isolated MPCs and an enriched population of pre-differentiated MPCs, wherein the EPCs and MPCs induce the formation of new blood vessels with functional connections to the subject's vasculature, and the formation of new adipose tissue. Tissue engineering techniques can be used for making vascularized adipose tissue in vivo. The engineered vascularized adipose tissue is needed in tissue repair, regenerative medicine, and reconstructive surgery and for cosmetic purposes. For example, engineered vascularized adipose tissue can be used to repair or reconstitute tissue loss due to severe burns, chronic wounds, injury, accident, disease, aging and surgery; the engineered vascularized adipose tissue can help provide proper shape and form to a subject who has significant tissue loss in parts of the body. The areas of the body where the engineered vascularized adipose tissue is useful include muscles, skin, limbs, face, and head. The engineered vascularized adipose tissues can be used in practically any body parts or area needing shaping, reconstruction or repair.

Tissue engineering is the use of a combination of cells, engineering and material methods, and suitable biochemical and physiochemical factors to improve or replace biological functions. Tissue engineering aims at developing functional cell, tissue, and organ substitutes to repair, replace or enhance biological function that has been lost due to congenital abnormalities, injury, disease, or aging, or repair fascia in hernias. The tissue that is engineered is used to repair or replace portions of or whole tissues. Often, the tissues involved require certain mechanical and structural properties for proper function. The term regenerative medicine is often used synonymously with tissue engineering, although those involved in regenerative medicine place more emphasis on the use of stem cells and progenitor cells to produce tissues and on promoting repair in situ. Tissue regeneration aims to restore and repair tissue function via the interplay of living cells, an extracellular matrix and cell communicators.

In one embodiment, the composition of enriched populations of EPCs and MPCs for neovascularization in newly formed adipose tissue and the promotion of vascularized adipose tissue formation are mixed with a filler material and both types of progenitor cells are delivered simultaneously to the subject.

In one embodiment, the filler material is an extracellular matrix, such as polymer scaffolds made of materials such as polyester based absorbable, hyaluronic acid, fibrin (from a combination of fibrinogen and thrombin), collagen, polyethylene glycol (PEG), silk, chemically modified alginate, HYLAFORM®, CAPTIQUE™ and PURAMATRIX™, a hydrogel-forming synthetic peptide.

In one embodiment, the filler material is biocompatible scaffold or matrix. In one embodiment, the filler material is a synthetic or natural material.

In one embodiment, a composition comprising EPCs/MPCs and/or pre-differentiated MPCs can be placed within a suitable biocompatible scaffold or matrix, and implanted to the subject. In one embodiment, a composition comprises 40% EPCs and 60% MPCs and/or pre-differentiated MPCs. In another embodiment, embryonic stem cells or other types of tissue-derived (parenchymal) cells can be used with the composition comprising EPCs/MPCs (e.g. 40%:60%) to seed a suitable biocompatible scaffold or matrix prior to implantation to the tissue repair location. Examples can be found in methods of constructing cardiac related structures described in U.S. Pat. Nos. 5,880,090, 5,899,937, 6,695,879, 6,666,886, 7,214,371, and US Pat. Publication No. 2004/0044403 and these publications are hereby incorporated by reference in their entirety.

In yet another embodiment, the composition comprising EPCs/MPCs and/or pre-differentiated MPCs disclosed herein can be ‘seeded’ into an artificial structure capable of supporting three-dimensional tissue formation. In one embodiment, the structure capable of supporting three-dimensional tissue formation is natural structure such as a de-celled body part, e.g., a de-celled piece of trachea, aorta, external ear or nose form cadavers. These structures, typically called scaffolds, are often critical, both ex vivo as well as in vivo, to recapitulating the in vivo milieu and allowing cells to influence their own microenvironments. Scaffold-guided tissue engineering involves seeding highly porous biodegradable scaffolds with cells and/or growth factors, followed by culturing the tissue engineering constructs in vitro for a time period. Subsequently the scaffolds are implanted into a host to induce and direct the growth of new tissue. The goal is for the cells to attach to the scaffold, then replicate, differentiate, and organize into normal healthy tissue as the scaffold degrades. This method has been used to create various tissue analogs including skin, cartilage, bone, liver, nerve, vessels, to name a few examples. The addition of the EPCs/MPCs mixture promotes the neovascularization of the tissue engineering constructs after implantation in the host.

In one embodiment, a biocompatible scaffold is used in tissue engineering. A scaffold fabricated from biocompatible materials is enveloped in a biocompatible material. The envelope of biocompatible material provides an improved substrate for cell attachment. In one embodiment, the biocompatible material used to envelope the scaffold is bioabsorbable. Suitable scaffolds include meshes, other filamentous structures, non-woven, sponges, woven or non-woven materials, knit or non-knit materials, felts, salt eluted porous materials, molded porous materials, 3D-printing generated scaffolds, foams, perforated sheets, grids, parallel fibers with other fibers crossing at various degrees, and combinations thereof. The core scaffold can be in a variety of shapes including sheets, cylinders, tubes, spheres or beads. The core scaffold can be fabricated from absorbable or non-absorbable materials. Suitable absorbable materials include glycolide, lactide, trimethylene carbonate, dioxanone, caprolactone, alklene oxides, ortho esters, polymers and copolymers thereof, collagen, hyaluronic acids, alginates, and combinations thereof. Suitable non-absorbable materials include, polypropylene, polyethylene, polyamide, polyalkylene therephalate (such as polyethylene therephalate polybutylene therephalate), polyvinylidene fluoride, polytetrafluoroethylene and blends and copolymers thereof. Suitable biocompatible materials that can be used to envelope the scaffold include absorbable or non-absorbable materials or a combination thereof. Suitable absorbable materials include those stated herein above. Suitable non-absorbable materials include those non-absorbable materials stated herein above. In some embodiments, the scaffold is embedded or encased in a bioabsorbable material.

In one embodiment, the filler material, matrix or scaffold has a stiffness property of about 1 Pa to 600 Pa. In other embodiments, the filler material has a stiffness property of about 5-520 Pa, 5-410 Pa, 10-450 Pa, 1-200 Pa, 1-100 Pa, 2-50 Pa, 2-75 Pa, 2-100 Pa, 2-150 Pa, 5-50 Pa, 5-75 Pa, 5-100 Pa, 5-150 Pa, 10-50 Pa, 10-75 Pa, 10-100 Pa or 10-150 Pa. The stiffness property of the filler/matrix/scaffold material, to a certain extent, affects the amount of neovascularization and vessel density at the implant site; there is an inverse correlation between stiffness property and the amount of neovascularization and vessel density. Generally, lower stiffness property of the filler/matrix/scaffold material tends to promote better or more neovascularization and vessel density (Allen P., J. Tiss. Eng. Regenative Med., 2011). Methods of determining rheological characteristics of extracellular matrices, gels and gel-like materials are known in the art. For example, the physical properties of each filler/matrix/scaffold material can be assessed using an AR2000 rheometer (TA Instruments) fitted with a 40 mm 4° cone. Each filler/matrix/scaffold material sample is reconstituted on ice, then 8004 and the temperature set to 37° C. Upon reaching full temperature, filler/matrix/scaffold material gels are allowed to polymerize if need, e.g., for 10 min for fibrin, 15 min for collagen and MATRIGEL™, and 20 min for PURAMATRIX™ gels. This polymerization time was dictated by the time required for G′ and G″ of each material to stabilize. Storage modulus, G′, and loss modulus, G″, as well as phase shift δ (such that tan δ=G′/G″) is assessed for each gel between 10⁻³ and 10⁻¹ strain units at 0.5 Hz oscillation frequency. Other methods are taught in “Rheological measurement” by A. A. Collyer (editor) and D. W. Clegg (editor), 2^{nd} edition, 1998, Publisher: Springer-Verlag New York.

One skilled in the art would be able to select a filler/matrix/scaffold material which can give sufficient support to the tissue while at the same time also promote sufficient neovascularization and vessel density in the newly formed adipose tissue thus formed.

Scaffolds can also be constructed from natural materials: in particular different derivatives of the extracellular matrix have been studied to evaluate their ability to support cell growth. Protein based materials, such as collagen or fibrin, and polysaccharidic materials, like chitosan or glycosaminoglycans (GAGs), have all proved suitable in terms of cell compatibility, but some issues with potential immunogenicity still remains. Among GAGs hyaluronic acid, possibly in combination with cross linking agents (e.g., glutaraldehyde, water soluble carbodiimide, etc.), is one of the possible choices as scaffold material. Functionalized groups of scaffolds may be useful in the delivery of small molecules (drugs) to specific tissues.

A variety of scaffolds and uses thereof are described in U.S. Pat. Nos. 6,103,255, 6,224,893, 6,228,117, 6,328,990, 6,376,742, 6,432,435, 6,514,515, 6,525,145, 6,541,023, 6,562,374, 6,656,489, 6,689,166, 6,696,575, 6,737,072, 6,902,932 and WO/2005/110050, they are hereby incorporated by reference in their entirety.

The procedures for tissue engineering the various tissue types can be found in the methods described in the examples herein, in Koji Kojima, et al., J. Thorac. Cardiovasc. Surg. 2002, 123:1177-1184, Duxbury M S, et al., Transplantation, 2004 77:1162-6, U.S. Pat. Nos. 5,700,289, 5,716,404, 6,123,727, 6,171,344, 6,503,273, 6,620,203, 6,666,886, 6,692,761, 6,656,489, 6,840,962, 6,737,053, 7,049,057, 7,049,139, 7,052,514, 7,052,518, 7,112,218, 7,179,287, 7,198,641 and these are hereby incorporated by reference in their entirety.

In one embodiment, the composition comprising EPCs/MPCs and/or pre-differentiated MPCs is directly implanted or injected to the site needing repair or reconstruction, for example, the part of the leg muscle that had undergone tissue resection to remove a tumor or the remaining chest area after breast mastectomy. Direct injection is useful for the repair of tissue, for example, muscles and the connective and support tissues such as ligaments, muscles, tendons and those tissues, such as the collagen-containing tissues which encapsulate organs, to name a few.

In one embodiment, the composition of EPCs/MPCs and/or pre-differentiated MPCs is implanted or contacted by direct injection to an adipose tissue. In another embodiment, the composition of progenitor cells is implanted or contacted by direct injection to areas of the body that require new adipose tissue to provide shape and form for the subject.

In some embodiments of the methods, compositions and kits described herein, the adipose tissue or the areas that are in need of new adipose tissue to give shape and form is found in areas selected from a group consisting of the skin, head, face, torso, muscle, legs and arms. In other embodiments, the areas that are in need of new adipose tissue to give shape and form can be any part of a subject's body.

In one embodiment, the composition of EPCs/MPCs and/or pre-differentiated MPCs can be used in craniofacial structure reconstruction. Craniofacial structures reconstruction requires the regeneration or de novo formation of dental, oral, and craniofacial structures lost to congenital anomalies, trauma, and diseases. Virtually all craniofacial structures are derivatives of mesenchymal cells. Biological therapies utilize mesenchymal stem cells, delivered or internally recruited, to generate craniofacial structures in temporary scaffolding biomaterials. Several craniofacial structures—such as the mandibular condyle, calvarial bone, cranial suture, and subcutaneous adipose tissue—have been engineered from mesenchymal stem cells. (J. J. Mao, et. al., J Dent Res 85(11):966-979, 2006) and is hereby incorporated by reference in its entirety.

The composition comprising EPCs/MPCs can be applied directly to wounds to stimulate wound healing and fill in the tissue lost as a result of the wound. Delivery can be direct injection to the wound, or to the adjacent tissue of the wound. For example, pressure ulcers, leg ulcers, abrasions, lacerations, incisions, donor sites and second degree burns on infected wounds, surgical incisions and traumatic wounds. The composition of EPCs/MPCs and/or pre-differentiated MPCs can be mixed with growth factors for promoting growth at the site of the wound, and the composition can be applied to the wound. The mixture can also be incorporated into a variety of wound dressing products such as wound dressing gauzes. The application of EPCs/MPCs and/or pre-differentiated MPCs with or without growth factors help promote healing in areas that may have a reduced capability of self-repair and renewal due to variety of medical conditions such as congestive heart failure, poor circulation, obesity, lymphatic obstructions and diabetes.

In one embodiment, provided herein is a composition for promoting in vivo neovascularization of a newly formed adipose tissue and for promoting in vivo vascularized adipose tissue formation; the composition comprises an enriched population of isolated EPCs, an enriched population of isolated MPCs and a pharmaceutically acceptable carrier. In one embodiment, composition further comprises an enriched population of pre-differentiated MPCs. In one embodiment, the composition comprising a composition of EPCs/MPCs is present in an amount sufficient to promote in vivo neovascularization at the site of implantation, for example, an open wound. In one embodiment, the composition further comprises pre-differentiated MPCs. In further embodiment, the pre-differentiated MPCs are differentiated towards the adipogenic lineage and not towards the osteogenic, muscle or chondrogenic lineage. In one embodiment, the pre-differentiated MPCs are differentiated to adipocytes.

In one embodiment, the EPCs comprise at least 10% but not more than 90% of the total cells in the composition. In another embodiment, the MPCs comprise at least 10% but not more than 90% of the total cells in the composition. In yet another embodiment, the EPCs comprise 40% and the MPCs comprise 60% of the total cells of the composition. In other embodiments, the EPC/MPC ratios can be 30:40, 30:50, 35:45, 35:50, 35:55, 35:60, 50:50, 30:60; 40:50, 45:55, 40:40, 45:45, and 45:50.

In one embodiment, the composition further comprises other cells that are non-EPCs and non-MPCs, e.g., other stem cells. In one embodiment, the composition further comprises smooth muscle cells. In one embodiment, the composition further comprises hematopoietic stem/progenitor cells. In some embodiments, these other cell types make up about 0%-30% of the total cells in the composition.

In one embodiment, the composition further comprises an extracellular matrix, such as polymer scaffolds made of materials such as polyester based absorbable, hyaluronic acid, collagen, polyethylene glycol (PEG), fibrin, silk, photo-crosslinkable gelatin methacrylate hydrogels, chemically modified alginate, HYLAFORM® and CAPTIQUE™ from Genzyme.

A pharmaceutically acceptable carrier is one that does not cause an adverse physical reaction upon administration and one in which maintains the viability of the EPCs/MPCs for delivery into the patient or use in tissue engineering. In one embodiment, the pharmaceutically acceptable carriers are inherently nontoxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol.

In one embodiment, other ingredients can be added to the composition, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the composition of a mixture of EPCs/MPCs should be sterile, is at a physiological pH of about 6 to about 8, and is isotonic to human bodily fluid.

In one embodiment, the composition comprising EPCs/MPCs and/or pre-differentiated MPCs, can include growth, differentiation, and/or angiogenesis factors that are known in the art to stimulated cell proliferation, differentiation, and angiogenesis the cells at the site where the composition is delivered. For examples, VEGF-A, bFGF, PDGF-BB, β-TGF and angiopoeitin-1.

The composition comprising EPCs/MPCs and/or pre-differentiated MPCs can be injected into the tissue repair site together with growth, differentiation, and angiogenesis factors that are known in the art to stimulated cell growth, differentiation, and angiogenesis in the appropriate cell type of the recipient tissue. In some embodiments, any one of these factors can be delivered to the implant site prior to or after the implant of the compositions described herein. Multiple subsequent delivery of any one of these factors can also occur to induce and/or enhance the neovascularization and adipogenesis. Suitable growth factors include but are not limited to transforming growth factor-beta (TGFβ), vascular endothelial growth factor (VEGF), platelet derived growth factor (PDGF), angiopoietins, epidermal growth factor (EGF), bone morphogenic protein (BMP), basic fibroblast growth factor (bFGF), insulin and 3-isobutyl-1-methylxasthine (IBMX). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag, and these are hereby incorporated by reference in their entirety.

In one embodiment, the composition can include one or more bioactive agents to induce healing or regeneration of damaged tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Other bioactive agents can promote increase mitosis for cell growth and cell differentiation.

A great number of growth factors and differentiation factors that are known in the art to stimulated cell growth and differentiation of the progenitor cells. Suitable growth factors and cytokines include any cytokines or growth factors capable of stimulating, maintaining, and/or mobilizing progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFβ, platelet derived growth factor (PDGF), angiopoeitins (Ang), epidermal growth factor (EGF), bone morphogenic protein (BMP), fibroblast growth factor (FGF), hepatocye growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α. Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment, the composition described is a suspension of progenitor cells in a suitable physiologic carrier solution such as saline. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In another embodiment, the composition described is a suspension of progenitor cells in gel-like components of the extracellular matrix. Components of the extracellular matrix comprise of fibrous proteins and polysaccharides, for example, glycosaminoglycans (GAGs), proteoglycans, heparan sulfate proteoglycans, chondroitin sulfate proteoglycans, keratan sulfate proteoglycans, hyaluronic acid, elastin, collagen, fibronectin, and laminin. In another embodiment, the composition of the invention is a suspension of progenitor cells in poly-lysine. The gel-like composition holds the progenitor cells in 3-dimensional space at the site of application on the tissue engineered construct or at the site of tissue repair. This prevents random diffusion of the cells and washing away of cells before they have a chance to adhere to the tissue engineered construct or tissue needing repair. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Examples of growth factors that can be used in a matrix comprising laminin, collagen IV and entactin, are EGF, bFGF, NGF, PDGF, IGF-1 and TGF-13. An example of such a gel-like composition is a matrix comprising laminin (56%), collagen IV (31%) and entactin (8%), EGF (0.5-1.3 ng/ml), bFGF (<0.1-0.2 pg/ml), NGF (<0.2 ng/ml), PDGF (5-48 pg/ml), IGF-1 (11-24 ng/ml), and TGF-β (1. 7.7 ng/ml).

In one embodiment, the quantity of progenitor cells (i.e., EPC-, MPCs and/or pre-differentiated MPCs) delivered in the composition disclosed herein to a tissue will vary based on the individual patient, the size of the construct or tissue or wound, the thickness of the construct, the number of sites for delivery within the tissue, wound, or adjacent tissue, the indication being treated and other criteria evident to one of ordinary skill in the art. Other factors to be taken into consideration when determining the precise of the amount of cells include the patient's weight, age, size of the treatment area, and the amount of time since injury. Additionally, the frequency of deliver also can vary. A therapeutically effective amount of progenitor cells in the composition is one sufficient to bring about neovascularization in a target organ, tissue or body area. In one embodiment, 1×10⁴ to 2×10¹² total progenitor cells are delivered in the composition. For engineering adipose tissues in vivo, at least 1×10⁶ total progenitor cells per 1 ml volume is recommended. The person of ordinary skill in the art can also readily determine the dosage of cells, amount of composition, type of pharmaceutically acceptable carrier and other bioactive agents to be delivered based on the present disclosure and the general knowledge known in the art.

The method of delivering the composition comprising EPCs and MPCs cells also vary based on the individual patient, the indication being treated and other criteria evident to one of ordinary skill in the art. The route(s) of delivery useful in a particular application are apparent to one of ordinary skill in the art. Routes of administration include, but are not limited to, topical, transdermal, and direct injection to the specific tissue site or organ. Topical and transdermal delivery is accomplished via a wound dressing impregnated with a composition of EPCs and MPCs, or the gel-like matrix suspension of progenitor cells, allowing the progenitor cells to migrate and enter the wound and also enter the blood stream. Direct injection delivery methods, including intramuscular and subcutaneous injections, can be accomplished using a needle and syringe, using a high pressure, needle free technique, like POWDERJECT™, constant infusion pump, a catheter delivery system, or the injection apparatus disclosed in the International Patent Publication number WO 2007112136.

In one embodiment, the total volume of the composition comprising EPCs and MPCs injected into tissue for therapeutic neovascularization is limited to 1 ml per injection site. The volumes injected can vary from the range of 50 μl to 1 ml. In one embodiment, several injection sites are selected within the tissue in need of neovascularization. This ensures even neovascularization of the target tissue or wound and promotes faster neovascularization. Volumes ranging from 50 μl to 1 ml can be injected at each site. Generally, the closer the sites of injection are together, the smaller the amount of the composition disclosed herein is delivered to each site. For example, at least 1×10¹² total cells are in a volume of 200 μl of composition for implanting. If a tissue in larger than 3 cm², the more than one injection volume of 200 μl can be injected to the tissue; about three injections can be placed, where each injection is of the volume of 200 μl. A physician skilled in the art can decide on the number of inject sites and the frequency of delivery depending on the tissue or wound needing vascularization. Example of direct localized delivery of therapeutics to the cardiac muscles is described in the International Patent Publication number WO 2007112136 and is hereby incorporated by reference in its entirety.

In one embodiment, the enriched populations of isolated EPCs, MPCs and/or pre-differentiated MPCs are delivered simultaneously to each site of delivery by any methods disclosed herein and known in the art. The EPCs, MPCs and/or pre-differentiated MPCs can be mixed in the recommended ratio as described herein and the mixture of progenitor cells is then delivered using a single needle and syringe at the injection site. Alternately, a multi-chambered needle-syringe, as described in the International Patent Publication number WO 2007112136, can be use for delivering the EPCs and MPCs simultaneously. Separate chamber holds a different progenitor cell type. When the syringe plungers are depressed, the different progenitor cell type enters a common chamber, and is mixed prior to delivery into the injection site. The depression of the syringe plunger can be automated to depress at different rates in order to achieve the recommended ratios of EPCs to MPCs as disclosed herein. In another embodiment, the enriched populations of isolated EPCs, isolated MPCs and/or pre-differentiated MPCs are delivered sequentially. Separate single-chambered needle-syringes can be used for delivery to a single injection site. It is a preferred embodiment that the EPCs, MPCs and/ pre-differentiated MPCs are pre-mixed in vitroprior to delivery to ensure the uniform distribution of the different cell types at the site of implant.

Encompassed in the invention is a kit that comprises an enriched population of isolated EPCs and an enriched population of isolated MPCs. In one embodiment, the kit further comprises a progenitor differentiation factor for pre-differentiating the MPCs in the kit. Examples of progenitor differentiation factors that induce MPCs to differentiate to adipocytes are dexamethasone, IBMX, insulin and indomethacin. In another embodiment, the kit further comprises an enriched population of pre-differentiated MPCs. In another embodiment, the MPCs are replaced with pre-differentiating MPCs in the kit.

In one embodiment, the kit further comprises at least one extracellular matrix or a biocompatible scaffold. Examples of extracellular matrix or biocompatible scaffold include but are not limited to polymer scaffolds made of materials such as polyester based absorbable, hyaluronic acid, collagen, polyethylene glycol (PEG), and chemically modified alginate.

In another embodiment, the kit further comprises at least one bioactive agent as disclosed herein to aid in cell growth, migration, and differentiation. In another embodiment, the kit further comprises an assortment of bioactive agents. In another embodiment, the kit also provides instructions for using the EPCs, MPCs, pre-differentiating MPCs, extracellular matrix, biocompatible scaffold, and bioactive agents to achieve neovascularization in tissues and organs, and formation of engineered adipose tissues.

In one embodiment of the methods, compositions and kits described herein, the enriched population of EPCs is at least 10% but not more than 90% of the total cells in the composition or kit.

In one embodiment of the methods, compositios and kits described herein, the enriched population of MPCs is at least 10% but not more than 90% of the total cells in the composition or kit.

In one embodiment of methods, compositions and kits described herein, the enriched population of EPCs is at least 40% of the total cells in the composition or kit.

In one embodiment of methods, compositions and kits described herein, the enriched population of EPCs is 40% of the total cells in the composition or kit.

In one embodiment, the composition further comprises pre-differentiated MPCs.

In one embodiment, the MPCs have been pre-differentiated in vitro. In one embodiment, the MPCs have been pre-differentiated to pre-adipocytes or adipocytes, particularly in the adipose lineage. An adipocyte also known as lipocytes and fat cell is a cell specialized for the storage of energy in the form of fat (consisting mainly of triglycerides); the fat is stored in a large cytoplasmic vesicle. A preadipocyte is the cell that is determined to become the fat cell or adipocyte. Preadipocytes can be induced by any method known in the art, e.g., by exposure to 5-azacytidine or 2′-deoxy-5-azacytidine drugs as described in Sager and Kovac, 1982, Proc. Natl. Acad. Sci. USA, 79:480-484. Preadipocytes distinguished from the progenitor MPCs by any method known in the art, e.g., (i) their inability to form other mesenchymal cell types; and (ii) their rapid accumulation of lipid in response to added insulin as described in Sager and Kovac, supra.

In one embodiment, the pre-differentiated MPCs at least 10% and not more that 90% of the total cells in the composition or kit.

In one embodiment, the MPCs and pre-differentiated MPCs collectively comprise at least 40% of the total cells in the composition or kit.

In one embodiment, the EPCs comprise about 40% and the MPCs and pre-differentiated MPCs collectively comprise 60% of the total cells of the composition or kit.

In one embodiment, the MPCs, EPCs and pre-differentiated MPCs are tested negative for HIV-1, HIV-2, HTLC-1, HTLV-2, Hepatitis B, Hepatitis C and mycoplasma.

The present invention can be defined in any of the following alphabetized paragraphs:
[A] A composition for use in promoting vascularized adipose tissue formation comprising an enriched population of isolated endothelial progenitor cells (EPCs) and an enriched population of isolated mesenchymal progenitor cells (MPCs), wherein the EPCs and MPCs induce the formation of new blood vessels with functional connections to a host vasculature and the formation of new adipose tissue.
[B] The composition of paragraph [A], wherein the vascularized adipose tissue is formed in vitro, in vivo or ex vivo.
[C] The composition of paragraph [A] or [B], wherein the new adipose tissue is differentiated from the MPCs.
[D] The composition of paragraph [A], [B] or [C], wherein the EPCs are derived from a source selected from a group consisting of bone marrow, cord blood, peripheral blood and blood vessel walls.
[E] The composition of paragraph [A], [B], [C] or [D], wherein the MPCs are derived from a source selected from a group consisting of amniotic fluid, bone marrow, cord blood, peripheral blood and adipose tissue.
[F] The composition of any one of paragraphs [A]-[E], wherein the progenitor cells are autologous to a recipient of the composition.
[G] The composition of any one of paragraphs [A]-[E], wherein the progenitor cells are allogenic and HLA type matched to a recipient of the composition.
[H] The composition of any one of paragraphs [A]-[G], wherein the progenitor cells are both obtained from a sample of peripheral blood.
[I] The composition of any one of paragraphs [A]-[H], wherein the composition of enriched populations of EPCs and MPCs are mixed with a filler material and the mixture of filler material and progenitor cells is delivered or implanted simultaneously.
[J] The composition of claim any one of paragraphs [A]-[I], wherein the filler material is an extracellular matrix.
[K] The composition of any one of paragraphs [A]-[J], wherein the extracellular matrix has a stiffness property of about 1 Pa to 600 Pa.
[L] The composition of any one of paragraphs [A]-[K], wherein the composition of progenitor cells is delivered or implanted into a subject.
[M] The composition of any one of paragraphs [A]-[L], wherein the composition of progenitor cells is delivered or implanted by direct injection to an adipose tissue.
[N] The composition of paragraph [M], wherein the implanted adipose tissue is found in areas selected from a group consisting of the skin, head, face, torso, muscle, legs, and arms.
[O] The composition of any one of paragraphs [A]-[N], wherein the enriched population of EPCs is at least 10% but not more than 90% of the cells in the composition.
[P] The composition of any one of paragraphs [A]-[O], wherein the enriched population of MPCs is at least 10% but not more than 90% of the cells in the composition.
[Q] The composition of paragraph [0], wherein the EPCs is at least 40% of the cells in the composition.
[R] The composition of any one of paragraphs [A]-[Q], wherein the composition further comprises pre-differentiated MPCs.
[S] The composition of paragraph [R], wherein the MPCs have been pre-differentiated in vitro.
[T] The composition of paragraph [R], wherein the MPCs have been pre-differentiated to pre-adipocytes and/or adipocytes.
[U] The composition of paragraph [R], [S] or [T], wherein the new adipose tissue is from the pre-differentiated MPCs.
[V] A method of promoting vascularized adipose tissue formation in a subject in need thereof comprising implanting a composition comprising an enriched population of isolated endothelial progenitor cells (EPCs) and an enriched population of isolated mesenchymal progenitor cells (MPCs), wherein the EPCs and MPCs induce the formation of new blood vessels with functional connections to the subject's vasculature and the formation of new adipose tissue.
[W] The method of paragraph [V], wherein the EPCs are derived from a source selected from a group consisting of bone marrow, cord blood, peripheral blood and blood vessel walls.
[X] The method of paragraph [V], wherein the MPCs are derived from a source selected from a group consisting of amniotic fluid, bone marrow, cord blood, peripheral blood and adipose tissue.
[Y] The method of any one of paragraphs [V]-[X], wherein the progenitor cells are autologous to a recipient.
[Z] The method of any one of paragraphs [V]-[X], wherein the progenitor cells are allogenic and HLA type matched to a recipient.
[AA] The method of any one of paragraphs [V]-[Z], wherein the progenitor cells are both obtained from a sample of peripheral blood.
[BB] The method of any one of paragraphs [V]-[AA], wherein the composition of enriched populations of EPCs and MPCs are mixed with a filler material and the progenitor cells are delivered simultaneously.
[CC] The method of any one of paragraphs [V]-[BB], wherein the filler material is an extracellular matrix.
[DD] The method of paragraph [CC], wherein the extracellular matrix has a stiffness property of about 1 Pa to 600 Pa.
[EE] The method of any one of paragraphs [V]-[DD, wherein the composition of progenitor cells is implanted by direct injection to an adipose tissue.
[FF] The method of paragraph [EE], wherein the adipose tissue is found in areas selected from a group consisting of the skin, head, face, torso, muscle, legs, and arms.
[GG] The method of any one of paragraphs [V]-[FF], wherein the enriched population of EPCs is at least 10% but not more than 90% of the cells in the composition.
[HH] The method of any one of paragraphs [V]-[GG], wherein the enriched population of MPCs is at least 10% but not more than 90% of the cells in the composition.
[II] The method of any one of paragraphs [V]-[HH], wherein the EPCs is about 40% of the cells of the composition.
[JJ] The method of any one of paragraphs [V]-[II], wherein the composition further comprise pre-differentiated MPCs, wherein the MPCs have been pre-differentiated in vitro.
[KK] The method of paragraph [JJ], wherein the MPCs have been pre-differentiated to pre-adipocytes or adipocytes.
[LL] A composition for promoting vascularized adipose tissue formation comprising an enriched population of isolated endothelial progenitor cells (EPCs); an enriched population of isolated mesenchymal progenitor cells (MPCs); an enriched population of pre-differentiated MPCs and a pharmaceutically acceptable carrier.
[MM] The composition of paragraph [LL], wherein the MPCs have been pre-differentiated to pre-adipocytes or adipocytes.
[NN] The composition of paragraph [LL] or [MM], wherein the EPCs are derived from a source selected from a group consisting of bone marrow, cord blood, peripheral blood and blood vessel walls.
[OO] The composition of paragraph [LL], [MM] or [NN], wherein the MPCs are derived from a source selected from a group consisting of amniotic fluid, bone marrow, cord blood, peripheral blood and adipose tissue.
[PP] The composition of any one of paragraphs [LL]-[OO], wherein the EPCs comprise at least 10% but not more than 90% of the total cells in the composition.
[QQ] The composition of any one of paragraphs [LL]-[PP], wherein the MPCs comprise at least 10% but not more than 90% of the total cells in the composition.
[RR] The composition of any one of paragraphs [LL]-[QQ], wherein the EPCs comprise about 40% of the total cells of the composition.
[SS] The composition of any one of paragraphs [LL]-[QQ], wherein the MPCs and pre-differentiated MPCs collectively comprise at least 40% of the total cells of the composition.
[TT] The composition of any one of paragraphs [LL]-[SS], wherein the EPCs comprise about 40% and the MPCs and pre-differentiated MPCs collectively comprise about 60% of the total cells of the composition.
[UU] The composition of any one of paragraphs [LL]-[TT], further comprising a filler material.
[VV] The composition of paragraph [UU], wherein the filler material is an extracellular matrix.
[WW] The composition of any one of paragraphs [LL]-[VV], wherein the extracellular matrix has a stiffness property of about 1 Pa to 600 Pa.
[XX] The composition of any one of paragraphs [RR]-[WW], wherein the progenitor cells are mixed with the filler material and the mixture of cells and filler material is delivered simultaneously.
[YY] A kit comprising a composition of any of paragraphs [LL]-[XX].
[ZZ] A kit comprising an enriched population of isolated endothelial progenitor cells (EPCs); an enriched population of isolated mesenchymal progenitor cells (MPCs); and at least one progenitor differentiation factor for pre-differentiating MPCs and/or EPCs.
[AAA] The kit of paragraph [ZZ], further comprising at least one filler material.
[BBB] The kit of paragraph [AAA], wherein the at least one filler material is an extracellular matrix or a biocompatible scaffold.
[CCC] The kit of paragraph [ZZ] or [BBB], wherein the at least one filler material has a stiffness property of about 1 Pa to 600 Pa.
[DDD] The kit of any one of paragraphs [ZZ]-[CCC] further comprising an enriched population of pre-differentiated MPCs.
[EEE] The kit of paragraph [DDD], wherein the MPCs have been pre-differentiated to pre-adipocytes or adipocytes.
[FFF] A method of promoting vascularized adipose tissue formation in a subject in need thereof comprising implanting a composition of any one of claims [LL]-[XX] or using the contents of a kit of any one of paragraphs [YY]-[EEE].
[GGG] The method of paragraph [FFF], wherein the progenitor cells are autologous to the subject recipient.
[HHH] The method of paragraph [FFF], wherein the progenitor cells are allogenic and HLA type matched to the subject recipient.
[III] The method of any one of paragraphs [FFF]-[HHH], wherein the progenitor cells are implanted by direct injection to an adipose tissue in the subject.
[JJJ] The method of paragraph [III], wherein the adipose tissue is found in areas selected from a group consisting of the skin, head, face, torso, muscle, legs, and arms.
[KKK] The method of any one of paragraphs [V]-[KK], [FFF]-[JJJ], further comprising selecting the subject for implanting.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Meolcular Biology, Vo. 203, 2003, Transgenic Mouse, editored by Marten H. Hofker and Jan van Deursen, which are all herein incorporated by reference in their entirety.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following example which should not be construed as limiting. The contents of all references cited throughout this application, as well as the figures are incorporated herein by reference.

### EXAMPLE

### Materials and Methods

### Cell Culture

Human blood-derived endothelial colony forming cells (h-ECFC) and bone marrow-derived mesenchymal stem cells (h-bmMSC) were isolated from human umbilical cord blood and bone marrow as previously described in Melero-Martin, J. M., et al. Circulation Research 103, 194-202 (2008). h-ECFCs are also known as the endothelial progenitor cells (EPCs) and human umbilical cord blood (cbEPCs).

h-watMSC were isolated by digesting human subcutaneous fat pads in a solution containing 1 mg/mL collagenase A and 2.5 U/mL dispase at 37° C. for 1 hour. After filtered through a 100-1 μm cell strainer, cell suspension was plated on uncoated tissue culture plates using MSCGM medium (Lonza, Walkersville, Md.) supplemented with 10% FBS, 1×GPS and 10 ng/mL bFGF (R&D, Minneapolis, Minn.). Unbound cells were removed at 48 hours, and the bound cell fraction maintained in culture until 70% confluence using MSCGM medium. ECFC were subcultured on 1% gelatin-coated tissue culture plates using ECFC-medium: EGM-2 (except for hydrocortisone; Lonza, Walkersville, Md.) supplemented with 20% FBS (Atlanta Biologicals, Lawrenceville, Ga.), 1× glutamine-penicillin-streptomycin (GPS; INVITROGEN™, Carlsbad, Calif.). h-bmMSC and h-watMSC were subcultured on uncoated tissue culture plates using MSCGM medium. Cells between passages 5 and 7 were used for all experiments.

### Retroviral Transduction of ECFCs

ECFCs were genetically labeled with red fluorescence proteins (DsRed) by retroviral infection with a pLVX-DsRed vector. Briefly, viruses were generated in HEK 293T with VIRAPOWER LENTIVIRAL packaging mix and LIPOFECTAMINE™ 2000 (INVITROGEN™). ECFCs (1×10⁶ cells at passage 1) were then incubated with 5 mL of virus stock for 6 hours in the presence of 12 μg/mL protamine sulfate (SIGMA-ALDRICH®). Puromycin-resistant transfected cells were selected with 2 μg/mL puromycin (INVITROGEN™) in regular culture medium. DsRed-ECFCs were characterized by immunofluorescent staining and expressed expected EC markers.

### Gel Composition (Per Implant)

The collagen/fibrin-based gel was made according to the method in Lin R. Z. and Melero-Martin J. M., 2011, J. Vis. Exp (Epub) and is incorporated herein by reference in its entirety. Basically, 200 μL collagen/fibrin-based solution comprised 3 mg/mL bovine collagen, 30 μg/mL human fibronectin, 25 mMHEPES, 10% 10×DMEM, 10% FBS, and 3 mg/mL fibrinogen; pH neutral. An aliquot of 50 uL of 10 U/mL thrombin was subcutaneously injected prior to cell mixture.

### Preparation of GelMA Hydrogels

GelMA was synthesized as described previously is J. W. Nichol, et al., (2010) Biomaterials, 31:5536. Briefly, type A porcine skin gelatin (Sigma-Aldrich) was dissolved in Dulbecco's phosphate buffered saline (DPBS; GIBCO) at 60° C. to make a uniform 10% (w/w) gelatin solution. Methacrylic anhydride (MA; SIGMA-ALDRICH®) was added to the gelatin solution at a rate of 0.5 ml/min under stirring conditions. Final concentration of MA was used 1% (v/v) (referred to herein as 1M GelMA). The mixture was allowed to react for 3 hours at 50° C. After a 5× dilution with additional warm DPBS, the GelMA solution was dialyzed against deionized water using 12-14 kDa cutoff dialysis tubes (Spectrum Laboratories) for 7 days at 50° C. to remove unreacted MA and additional by-products. The dialyzed GelMA solutions were frozen at −80° C., lyophilized and stored at room temperature. Before use, a GelMA prepolymer solution was prepared by dissolving the freeze-dried GelMA (0.5 w/v % final) and the photoinitiator (Irgacure 2959, 0.5 w/v %; CIBA Chemicals) in DPBS at 80° C. Photocrosslinking was achieved by exposing the GelMA prepolymer to 6.7 mW/cm² UV light (360-480 nm; using OmniCure S2000 UV lamp; Lumen Dynamics) for 20 seconds at room temperature. Additional method of transdermal photopolymerization of GelMA can be found in Elisseeff et al, 1999, PNAS, 96: 3104-3107, the reference is incorporated herein by reference in its entirety.

### Adipogenesis Assay

Confluent MSCs were cultured for 10 days in DMEM low-glucose medium with 10% FBS, 1×GPS, and adipogenic supplements (5 μg/mL insulin, 1 μM dexamethasone, 0.5 mM isobutylmethylxanthine, 60 μM indomethacin, 1 μM Rosiglitazone). Differentiation into adipocytes was assessed by Oil Red O staining as previously described in Melero-Martin, J. M., et al. Circulation Research 103, 194-202 (2008).

### Osteogenesis Assay

Confluent MSCs were cultured for 21 days in DMEM low-glucose medium with 10% FBS, 1×GPS, and osteogenic supplements (1 μM dexamethasone, 10 mM (3-glycerophosphate, 60 μM ascorbic acid-2-phosphate). Differentiation into osteocytes was assessed by von kossa staining (Calcium Stain Kit, Diagnostic BioSystems).

### Chondrogenesis Assay

Suspensions of MSCs were transferred into 15 ml polypropylene centrifuge tubes (500,000 cells/tube) and gently centrifuged. The resulting pellets were statically cultured in DMEM high-glucose medium with 1×GPS, and chondrogenic supplements (1× insulin-transferrin-selenium, 1 μM dexamethasone, 100 μM ascorbic acid-2-phosphate, and 10 ng/mL TGF-β1). After 21 days, pellets were fixed in 10% buffered formalin overnight, embedded in O.C.T., and sectioned (8 μm-thick) using a cryostat microtome. Differentiation into chondrocytes was assessed by evaluating the presence of glycosaminoglycans after Alcian Blue staining as previously described in Melero-Martin, J. M., et al. Circulation Research 103, 194-202 (2008).

### Flow Cytometry

Cytometry analyses were carried out by labeling cells with phycoerythrin (PE)-conjugated mouse anti-human CD31 (Ancell, Bayport, Minn.), PE-conjugated mouse anti-human CD90 (BD Pharmingen, San Jose, Calif.), fluorescein isothiocyanate (FITC)-conjugated mouse anti-human CD45 (BD Pharmingen), FITC-conjugated mouse IgG1 (BD Pharmingen), and PE-conjugated mouse IgG1 (BD Pharmingen) antibodies (1:100 dilution). For in vivo studies, retrieved cells were analyzed using the following additional antibodies: PE-Cy5-conjugated rat anti-mouse CD45 (BD Pharmingen), PE-conjugated mouse anti-human CD90 (BD Pharmingen), FITC-conjugated mouse anti-human CD31 (BD Pharmingen), PE-conjugated rat anti-mouse CD29 (eBioscience, San Diego, Calif.) and APC-conjugated rat anti-mouse CD31 (eBioscience). Antibody labeling was carried out for 20 minutes on ice followed by 3 washes with PBS/1% BSA/0.2 mM EDTA and resuspension in 1% paraformaldehyde in PBS. Flow cytometry analyses were performed using a Becton Dickinson FACScan flow cytometer and FlowJo software (Tree Star Inc., Ashland, Oreg.).

### Mice

Mice were housed in compliance with Children's Hospital Boston guidelines, and all animal-related protocols were approved by the Institutional Animal Care and Use Committee. Six-week-old male athymic nu/nu mice (Massachusetts General Hospital, Boston, Mass.) and GFP—SCID mice (kindly provided by Taturo Udagawa, Children's Hospital Boston) were used for all experiments.

### In Vivo Vasculogenesis Assay

The formation of vascular networks in vivo was evaluated using a xenograft model of transplantation into immunodeficient mice (P. Au, et al. 2008, Blood, 111:1302; D. O. Traktuev, et al., 2009, Circ. Res. 104:1410). Briefly, 5×10⁵ DsRed-ECFC and 5×10⁵ MSC were embedded in 200 μl hydrogel and crosslinked as described. Cell-laden hydrogels were incubated for 24 hours in EGM-2 medium before implantation. Next day, hydrogel constructs were surgically implanted into the subcutaneous space on the back of six-week-old male nu/nu mice. Rat-tail type 1 collagen gel (3 mg/mL in DPBS; pH 7.4) containing the same amount of cells served as control. One construct was implanted per mouse. Each experimental condition was performed with 7 mice.

In vivo vasculogenic and adipogenic assay. Briefly, a total of 2×10⁶ cells was resuspended in 200 μl of ice-cold MATRIGEL™, at a ratio of 40:60 (ECFC:MSC). Control groups included implants that contained ECFC alone and MSC alone. The cell mixture was subcutaneously injected into the upper dorsal region of the mouse using a 26-gauge needle. One implant was injected per mouse. Unless otherwise stated, each experimental condition was performed with 4 mice. The length of each in vivo experiment varied from 7 days to 4 weeks depending on the study. Implants as well as adipose tissue were carefully harvested and photographed at the end of each experiment, and they were subsequently preserved for histological examination. When the compositions comprising EPCs and MPCs were injected into the mice, the composition was referred to as an implant. However, when the implants were excised from the mice for vasculogenic and adipogenic analysis, the implants were referred to as explants.

### Histology and Immunohistochemistry

Mice were euthanized at different time points and MATRIGEL™, collagen/fibrin-based or GelMA explants were removed, fixed in 10% buffered formalin overnight, embedded in paraffin, and sectioned (7-μm-thick sections). Standard Hematoxylin and Eosin (H&E) staining was performed at the Dana-Farber Histopathology Core Center. For immunohistochemistry, sections were deparaffinized, and antigen retrieval was carried out by heating the sections in Tris-EDTA buffer (10 mM Tris-Base, 2 mM EDTA, 0.05% Tween-20, pH 9.0). The sections were blocked for 30 minutes in 5-10% blocking serum and incubated with primary antibodies for 1 hour at room temperature. The following primary antibodies were used: For MATRIGEL™ explants, rabbit anti-gfp (1:2000; ABCAM®) and goat anti-perilipin-A (1:100; ABCAM®) antibodies. Secondary antibodies incubations were carried out for 1 hour at room temperature using FITC- or TexasRed-conjugated antibody (1:200; Vector Laboratories) after incubation with the respective primary antibody. For GelMA explants, mouse anti-human CD31 (for human microvessel detection; 1:50; DakoCytomation, Clone JC70A) and mouse anti-SMA (for perivascular cell detection; 1:200; SIGMA-ALDRICH®, Clone 1A4). For human CD31 immunohistochemistry, horseradish peroxidase conjugated mouse secondary antibody (1:200; Vector Laboratories) and 3,3′-diaminobenzidine (Vector Laboratories) were used, followed by hematoxylin counterstaining and Permount mounting. For SMA immunofluorescence, secondary antibody incubations were carried out for 1 hour at room temperature using Alexa Fluor 488-conjugated anti-mouse secondary antibody (1:200; INVITROGEN™). Rhodamine-labeled Ulex Europaeus Agglutinin I (UEA-1; 1:100; Vector Laboratories) was used to detect human microvessels. All the fluorescent-stained sections were counterstained with DAPI.

### Microvessels Density (MVD)

For the assessment of MVD, ten random fields from mid-H&E sections of each of the explants in the group were quantified by counting luminal structures containing red blood cells. MVD is reported as the average number of erythrocyte-filled microvessels from the fields analyzed and expressed as vessels/millimeter². Values reported for each experimental condition were mean values±S.D. obtained from seven individual mice.

### Adipose Tissue Evaluation

Sections obtained from subcutaneous implants were stained with H&E and examined under a light microscope. The fraction of the area occupied by adipocytes in each implant was calculated using an image analysis algorithm (ImageJ software; NIH, Bethesda, Md.) based on the evaluation of images corresponding to H&E-stained sections taken from the middle part of the implants. Briefly, adipocytes were first identified and marked based on both their white histological color and defined circularity. Then, pictures were converted into binary (black and white) with the area occupied by the identified adipocytes converted into black and the rest of the area processed as white. Finally, each binary-converted image was analyzed and both the number of adipocyte and the area of each individual adipocyte quantified. The fraction of implant (or native tissue) occupied by adipocytes was calculated by dividing 1) the total area occupied by adipocytes by 2) the total area of each section. Additionally, the number of adipocytes per mm² of implant and the average area occupied by each adipocyte (μm²) were calculated. Mouse subcutaneous adipose tissues were used as positive control.

### Microscopy

Phase microscopy images were taken with a Nikon Eclipse TE300 inverted microscope (Nikon, Melville, N.Y.) using Spot Advance 3.5.9 software (Diagnostic Instruments, Sterling Heights, Mich.) and a 10×/0.3 objective lens. All fluorescent images were taken with a Leica TCS SP2 Acousto-Optical Beam Splitter confocal system equipped with DMIRE2 inverted microscope (Diode 405 nm, Argon 488 nm, HeNe 594 nm; Leica Microsystems, Wetzlar, Germany) using a 63×/1.4 oil objective lens. Non-fluorescent images were taken with a Primo star microscope (Zeiss, Oberkochen, Germany) equipped with AxioCam MRc camera (Zeiss) using a 40×/0.6 objective lens.

### Statistical Analyses

One-way analysis of variance was performed and confirmed with two-tailed paired Student's t test using GraphPad Prism 4 software. Data are reported for each experimental condition as mean values±S.D. of the mean. P values less than 0.05 were considered significant.

### Results

### Example 1

### Neovascularization Regulates De Novo Adipogenesis

Here, the inventors show a progenitor cell-based approach to sequentially promote vasculogenesis and adipogenesis in vivo.

Endothelial progenitor cells from human umbilical cord blood (cbEPCs) and white adipose tissue (wat)-derived mesenchymal stem cells from GFP-057BL/6 mice (gfp−watMSCs) were isolated. The isolated cells were expanded in culture and characterized prior to their use. To test their differentiation ability in vivo, MATRIGEL™ implants (200 μL) containing cbEPCs and gfp−watMSCs (40:60) were injected subcutaneously into immunodeficient mice. MATRIGEL™ implants were harvested at different time points and the presence of both new blood vessels and adipocytes were evaluated. Control implants were generated using gfp−watMSCs alone.

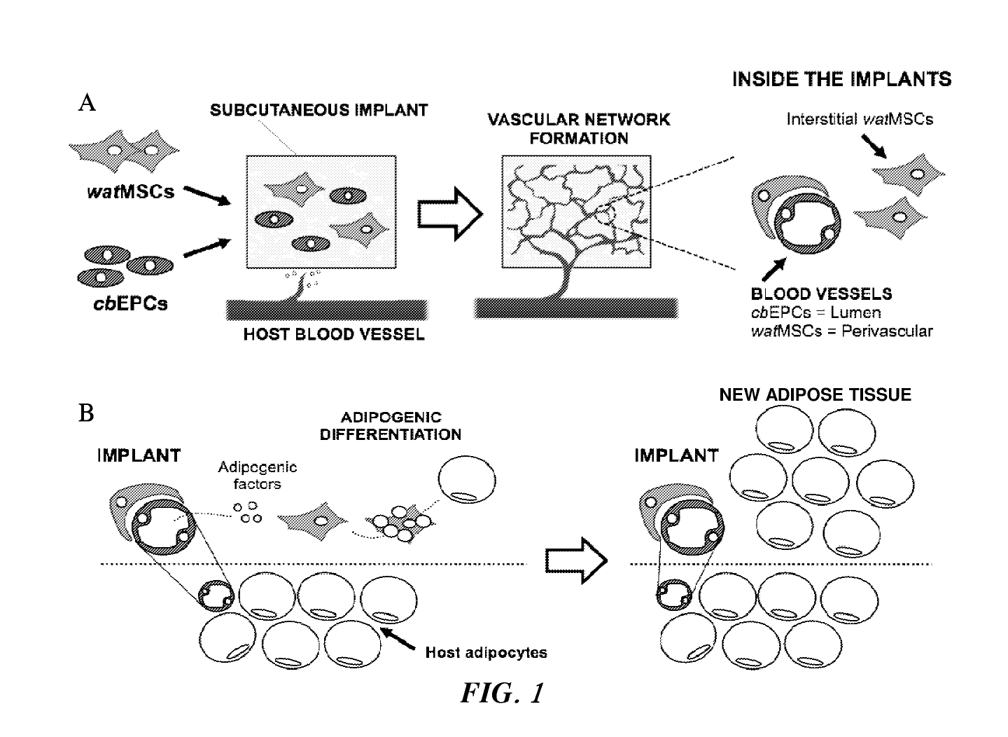

FIG. 1 shows the general plan for vasculogenesis and adipogenesis. Purified and defined adipose tissue-derived watMSCs and cord blood-derived cbEPCs are used to drive adipose tissue development in vivo. Step-1 shows the implantation of watMSCs and cbEPCs to generate a vascular network that connects to the host vasculature. Step-2 shows that vascularization induces watMSCs to undergo adipogenesis, adopting the phenotype of the surrounding tissue at the site of implantation.

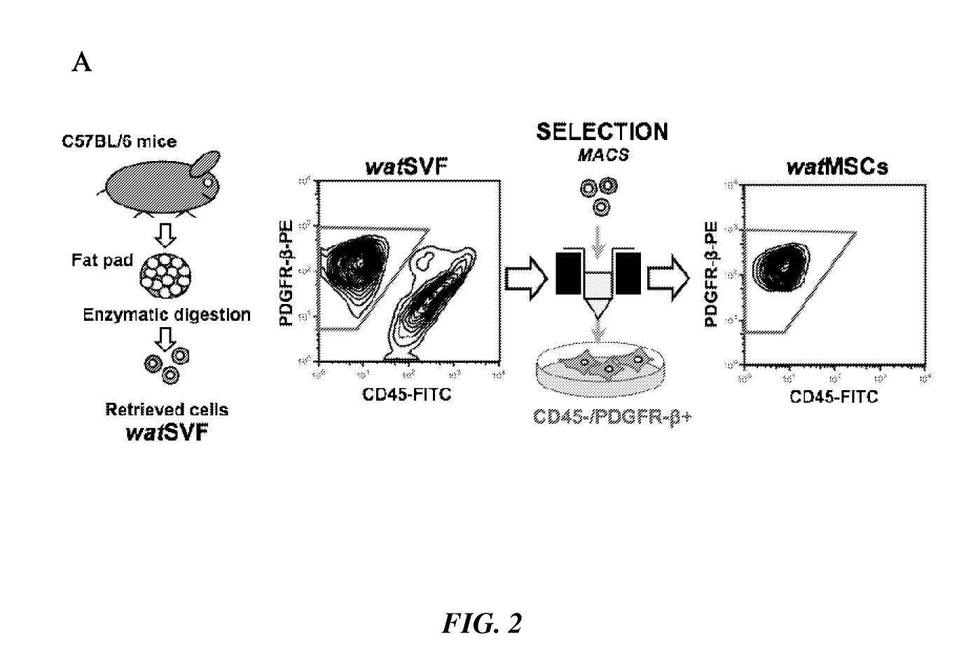

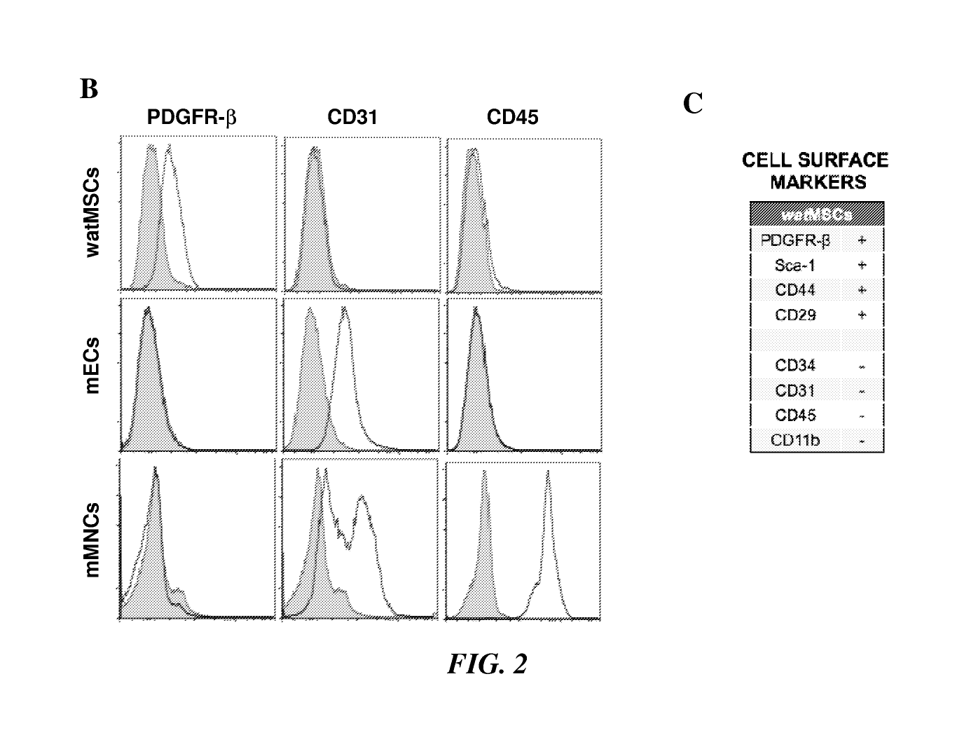

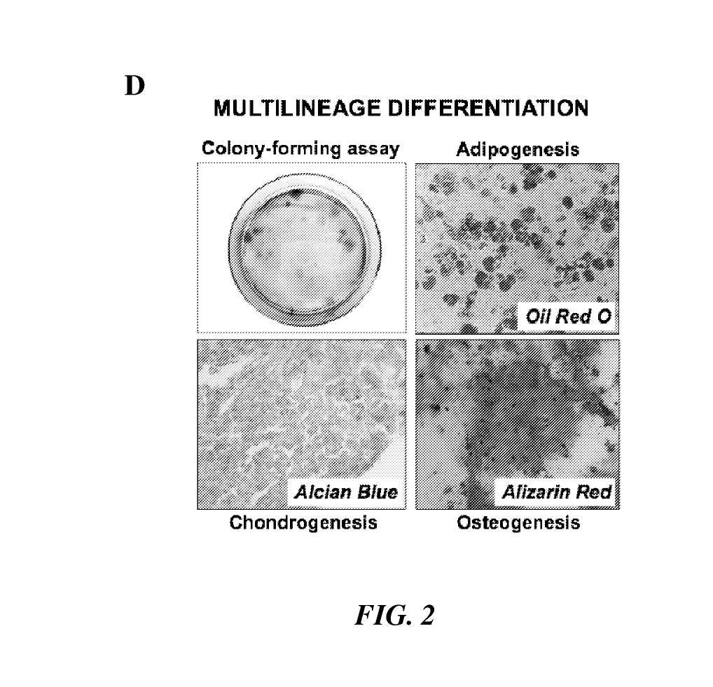

FIG. 2 shows the isolation and characterization of watMSCs. Subcutaneous fat pads were removed from C57BL/6 mice, minced, and digested (collagenase/dispase). Mature adipocytes were removed and the stromal-vascular fraction (watSVF) obtained by centrifugation. Hematopoietic cells were depleted using magnetic beads coated with an antibody against CD45. watMSCs were then obtained by positive selection of PDGFR-β expressing cells. FACS analysis of watMSCs revealed positive expression of PDGFR-β, Sca-1, CD44, and CD29. watMSCs were negative for endothelial cell surface markers (CD31, CD34) and hematopoietic cell markers (CD45, CD11b). watMSC displayed colony-forming ability and multilineage differentiation capability along mesenchymal lineages (adipocytes, chondrocytes, osteocytes).

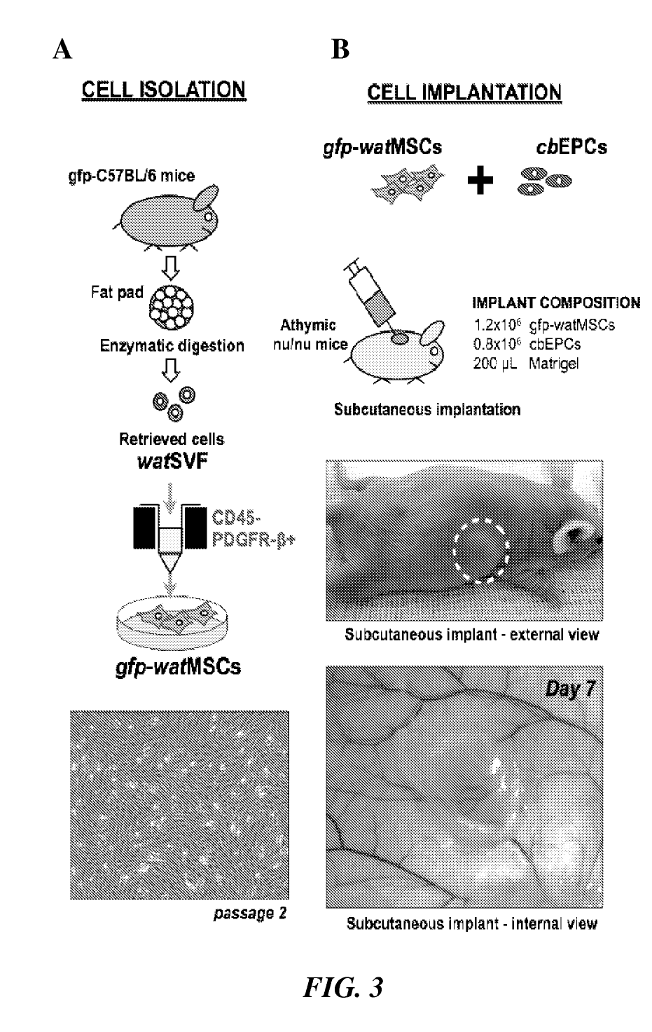

FIG. 3 shows the implantation experiment in vivo. Subcutaneous fat pads were resected from gfp−057BL/6 mice and the watSVF obtained after removal of mature adipocytes. gfp−watMSCs were selected (CD45−/PDGFR-β+) from the watSVF using antibody-coated magnetic beads (MACS) and expanded in culture for 3 passages. gfp−watMSCs were combined with human cord blood-derived endothelial progenitor cells (cbEPCs) and the mixture resuspended in MATRIGEL™ and injected subcutaneously into immunodeficient nu/nu mice. Mice were euthanized at either 1 or 4 weeks and retrieved implants were analyzed histologically.

At one week, (see FIG. 4), implants revealed numerous human vessels containing murine erythrocytes confirming these functional blood vessels were lined by the implanted cbEPCs. gfp−watMSCs were found in a perivascular position and expressed α-smooth muscle cell actin (α-SMA), indicating an early role as pericytes.

Histological examination of implants at 1 week revealed the presence of an extensive vascular network that stained positive for human CD31 (hCD31). The microvessel density of implants containing cbEPCs was significantly higher than those implants with only watMSCs (P<0.05; n=4). gfp+watMSCs were found both interstitially distributed inside the implant and in perivascular locations serving as pericytes (α-SMA+).

Subcutaneous implants were resected at 4 weeks and the presence of adipose tissue was histologically examined and quantified. At four weeks, vascularized implants contained numerous gfp+ adipocytes (perilipin-A+, both uni- and multilocular), indicating that the implanted gfp−watMSCs underwent adipogenic differentiation. Both microvessel density at one week and the number of gfp+ adipocytes at four weeks were significantly higher compared to control implants with gfp−watMSCs alone, indicating an important role for cbEPCs during adipogenesis.

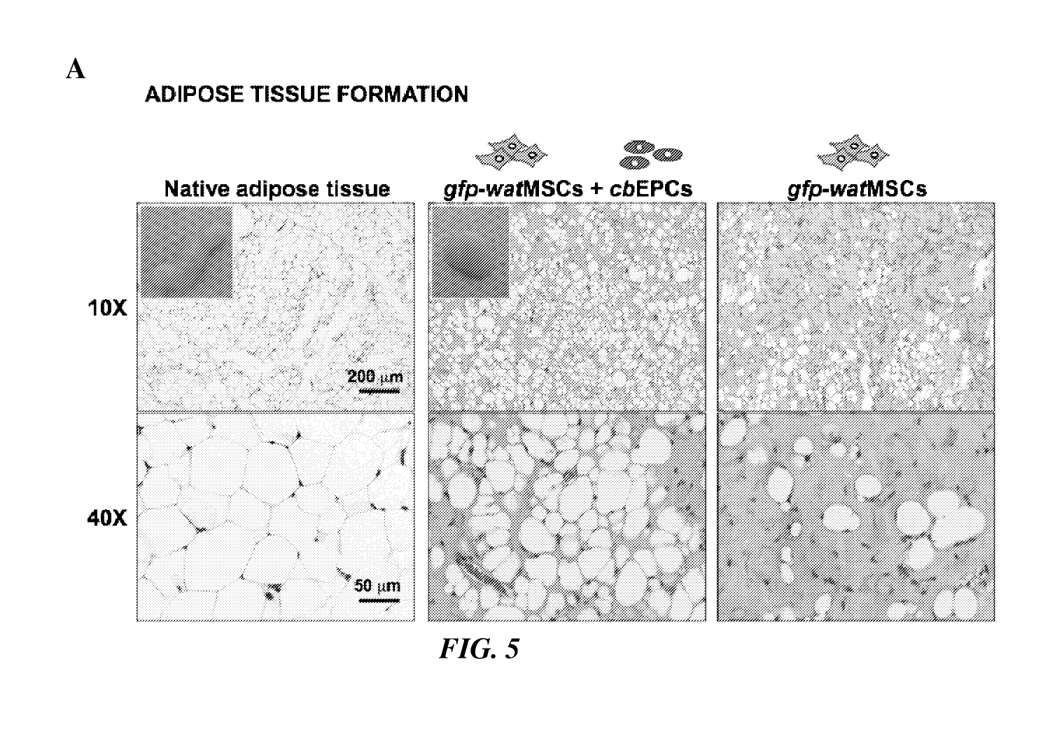

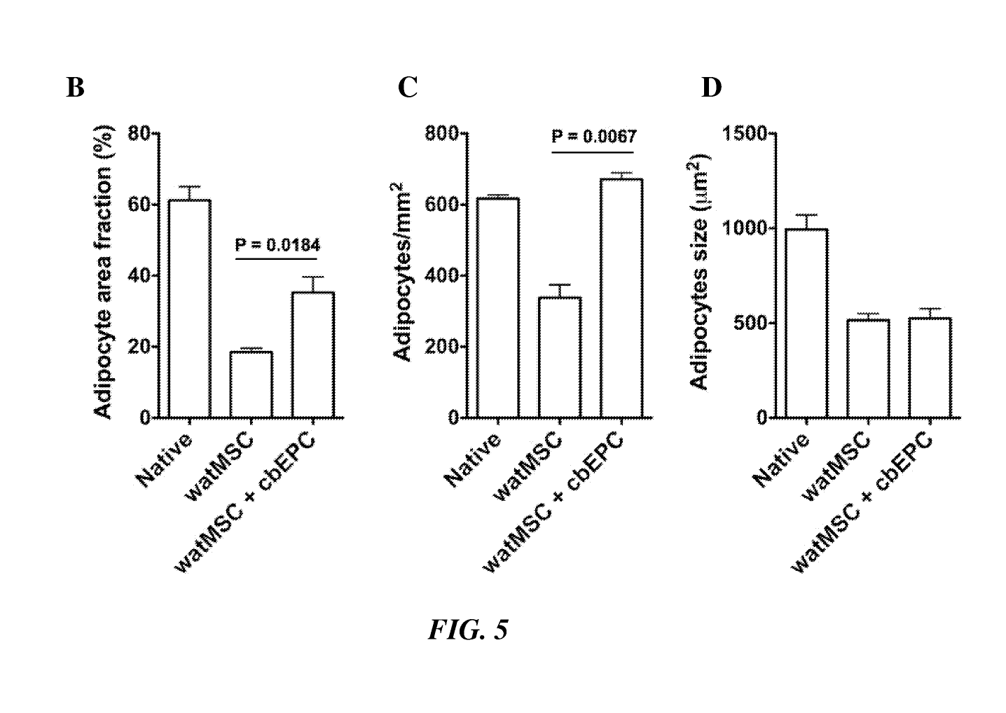

Implants containing watMSCs/cbEPCs presented significantly larger fraction of adipose tissue than those containing watMSCs alone, indicating a beneficial effect of high initial microvascular density on later adipogenesis. This larger presence of adipose tissue was due to a higher density of adipocytes rather than a difference in the average adipocyte size. The large majority of adipocytes found in the implants were gfp+watMPCs that had differentiated in vivo (gfp+, perilipin-A+), in contrast with host adipocytes (gfp−, perilipin-A+) that could be seen in the subcutaneous adipose tissue adjacent to the implant (FIG. 5).

### Example 2

### In Vivo Co-Cultures of ECFCs and Human watMSCs in MATRIGEL™

Characterization of Human Mesenchymal Stem Cells (h-MSC)

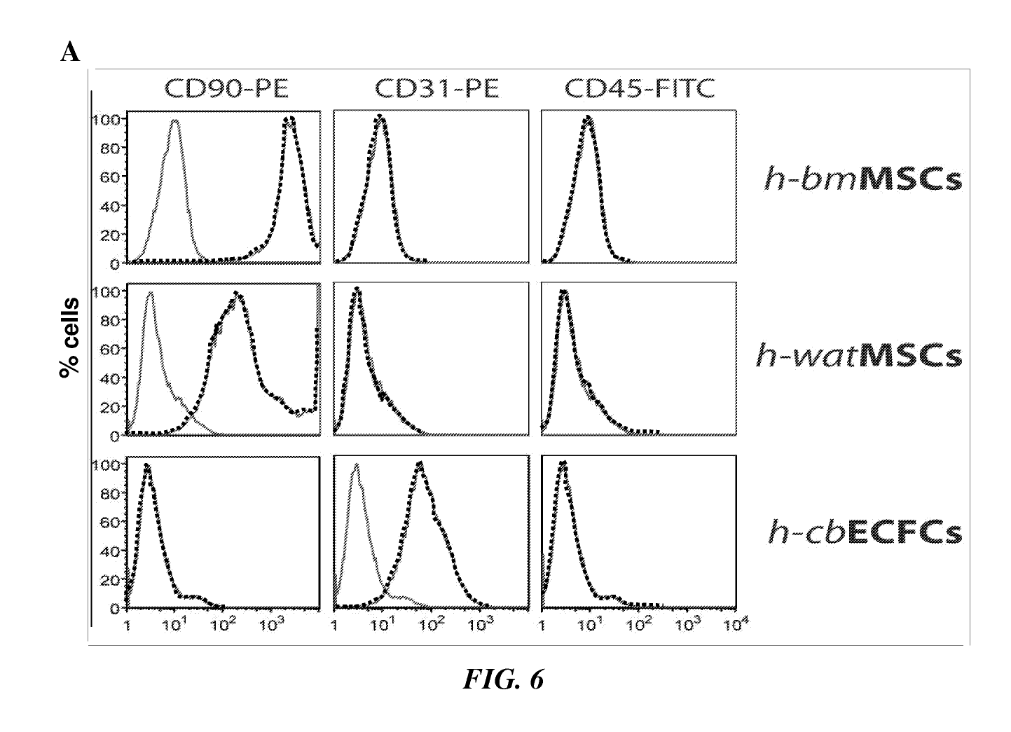

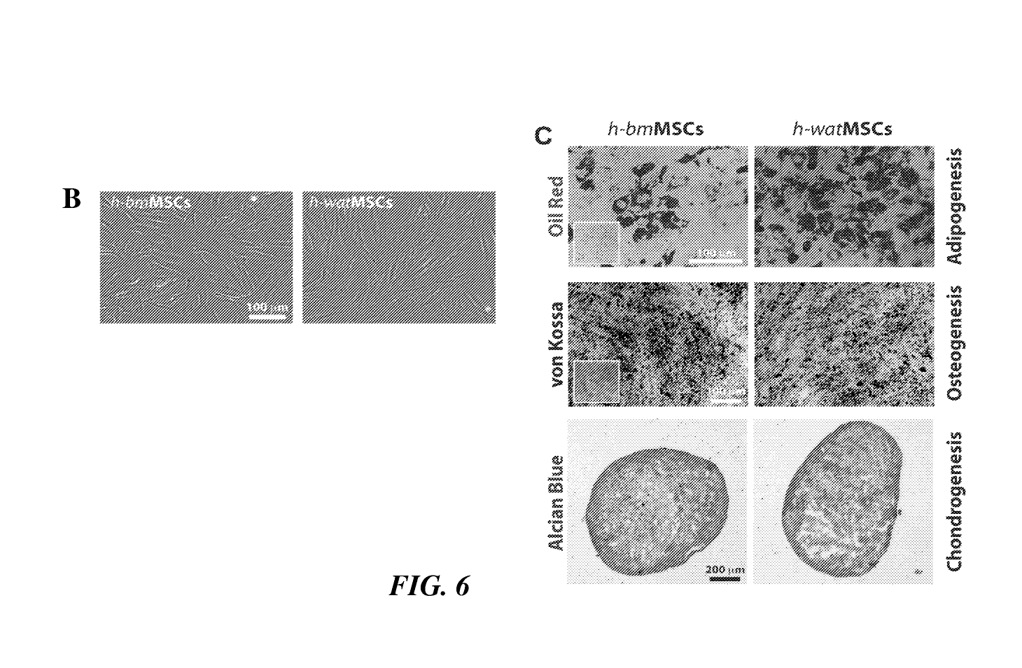

The inventors successfully isolated human mesenchymal stem cells (hMSCs) and characterized them prior to use in the various vasculogenic and adipogenic experiments. The MSC from various sources: bone marrow (h-bmMSCs), white adipose tissue (h-watMSCs) and code blood (h-cbMSCs), and ECFC were characterized by their cell surface markers as well as the ability of these cells to differentiate into the cell types that are typical of the mesenchymal lineage, e.g., bone, cartilage and fat cells. Flow cytometry analysis of h-bmMSC, h-watMSC, and human cord-blood derived endothelial colony-forming cells (h-cbECFC) for the mesenchymal marker CD90, the endothelial marker CD31, and hematopoietic marker CD45 is shown in FIG. 6A. Dotted lines represent cells stained with fluorescent antibodies. Isotype-matched controls are overlaid in a solid line on each panel. As expected, both h-bmMSCs and h-watMSCs expressed h-CD90 but did not express h-CD31 or h-CD45. The typical spindle morphology characteristic of MSC in culture is shown in FIG. 6B. FIG. 6C is supporting evidence that the isolated human MSCs have their multilineage differentiation capacity as expected of mesenchymal stem cells. Both h-bmMSCs and h-watMSCs were differentiated into 1) adipocytes (adipogenesis) as assessed by oil red 0 staining (inset represents negative control in non-inducing media); 2) osteocytes (osteogenesis) as revealed by von kossa staining (insets represent negative control in non-inducing media); and 3) chondrocytes (chondrogenesis) as revealed by the presence of glycosaminoglycans, detected by Alcian blue staining.

Accordingly, the inventors demonstrated that human MSCs can be isolated from human bone marrow, human white adipose tissues and human code blood, and that the isolated cells, regardless of source have the expected morphology, express the expected markers, and can undergo multi-lineage differentiation in culture.

Implantation of Human-Derived MSC in Combination with ECFC is Beneficial for MSC Engraftment and Survival

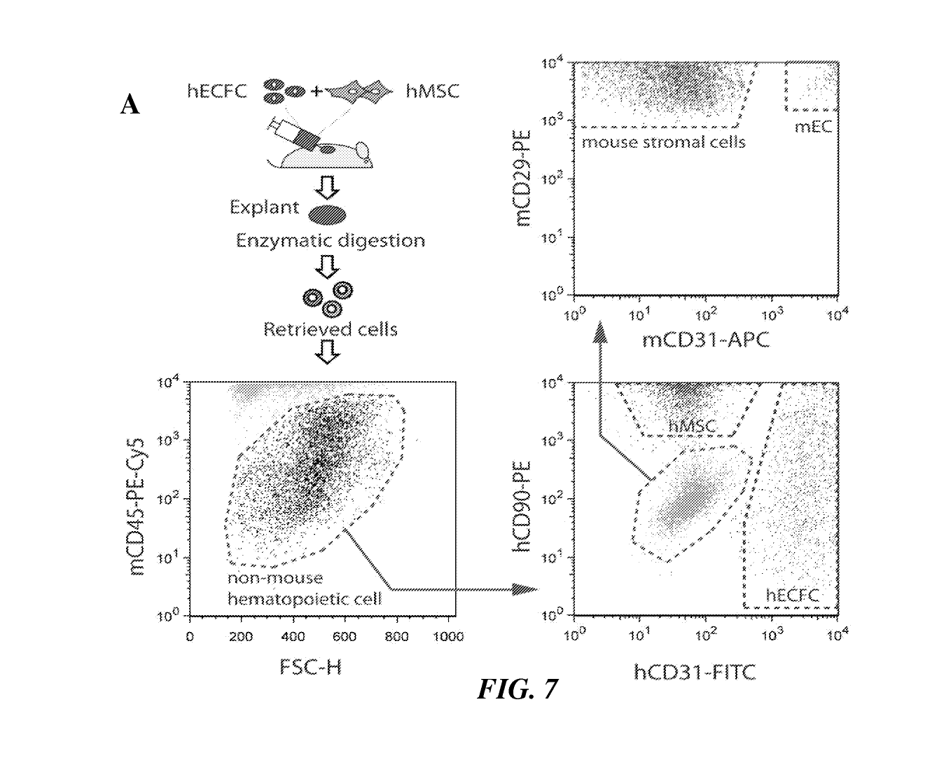

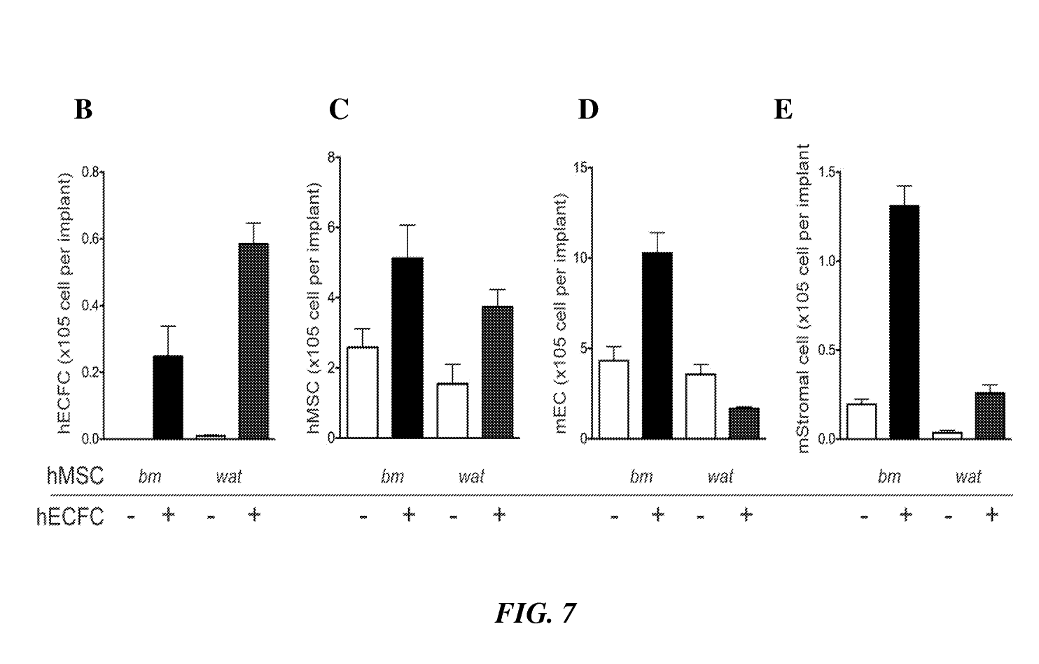

A total of 1.2×10⁶ h-MSCs were resuspended in 200 μL of MATRIGEL™ in the presence or absence of 0.8×10⁶ h-ECFC. The mixture was implanted on the back of 6-week-old nu/nu mouse by subcutaneous injection. The general protocol of the implantation experiment is shown in FIG. 7A. Implants were harvested after 7 days, enzymatically digested, and the retrieved cells were analyzed by flow cytometry using antibodies against murine CD45 (mCD45−PE-Cy5), human CD90 (h-CD90−PE), human CD31 (h-CD31−FITC), mouse CD29 (m-CD29−PE), and mouse CD31 (mCD31−APC). The following cell populations were identified and quantified in n=4 replicates: hECFC that is mCD45− and hCD31+ (in FIG. 7B), hMSC that is mCD45−, hCD31−, hCD90+ (in FIG. 7C), mouse endothelial cells, mEC that is mCD45−, hCD31, hCD90−, mCD31+ (in FIG. 7D), and mouse stromal cells that is mCD45−, hCD31−, hCD90−, mCD31−, mCD29+ (in FIG. 7E).

The data obtained shows that when implantation of h-MSCs is done in combination with h-ECFC, the number of engrafted h-MSC after 7 days in vivo is significantly higher than in the absence of h-ECFC (FIG. 7C). This was true for both h-bmMSCs and h-watMSCs.

Implants Containing h-watMSC and ECFC have More Blood Vessels than in the Absence of h-ECFC

A total of 1.2×10⁶ h-watMSCs were resuspended in 200 μL of MATRIGEL™ in the presence or absence of 0.8×10⁶ h-ECFC. The mixture was implanted on the back of 6-week-old GFP-expressing SCID mouse by subcutaneous injection. The general protocol of the implantation experiment is shown in FIG. 8A. Implants were harvested after 7 days. Macroscopic view of an explant at day 7 is depicted. H&E staining of sections taken from the explants at day 7 revealed the presence of numerous blood vessels containing murine erythrocytes (FIG. 8B). Microvessel densities of implants harvested at day 7 were also quantified by counting lumenal structures containing erythrocytes. The groups evaluated were implants containing 1) h-ECFC alone, 2) h-watMSC alone, and 3) h-watMSC+h-ECFC (FIG. 8C). This data show that when implantation of h-watMSCs is done in combination with h-ECFC, the number of blood vessels observed inside the implant at day 7 is significantly higher than in the absence of h-ECFC. Implanting h-watMSC alone produces blood vessels, but fewer and also these blood vessels are all mouse vessels that infiltrate the implant. On the other hand, h-watMSCs+h-ECFC produces more vessels, and these vessels are now a combination of human vessels (lumens lined by h-ECFCs) and mouse infiltrated vessels.

Implants Containing h-watMSC and h-ECFC have More Adipocytes than in the Absence of h-ECFC

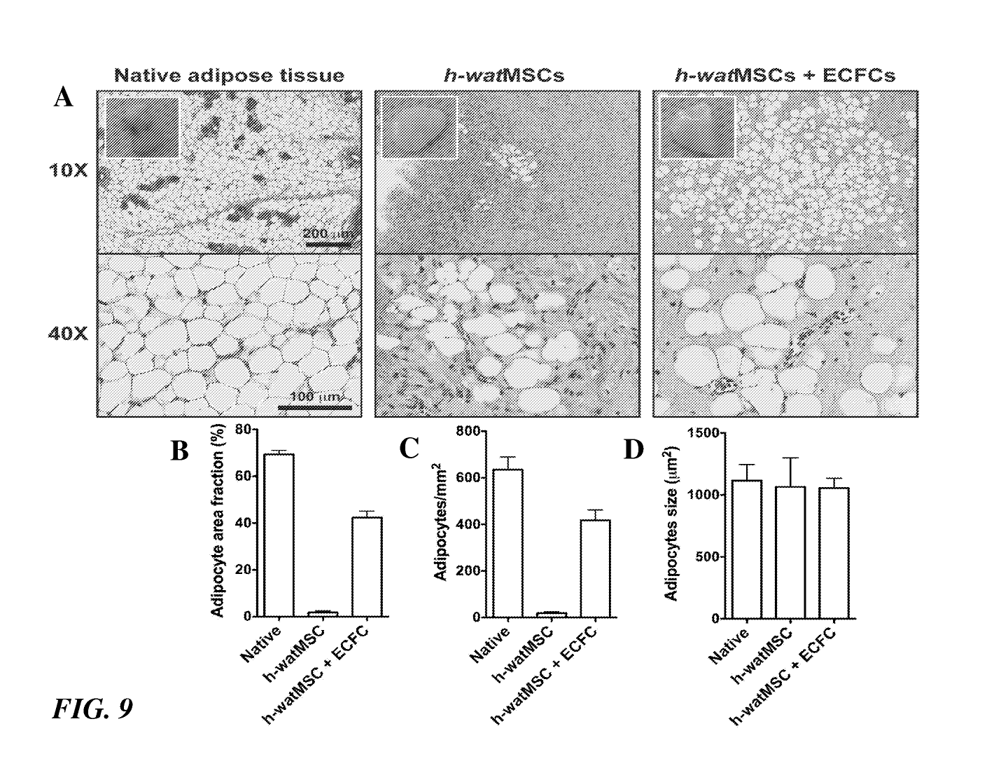

A total of 1.2×10⁶ h-watMSCs were resuspended in 200 μL of MATRIGEL™ in the presence or absence of 0.8×10⁶ h-ECFC. The mixture was implanted on the back of 6-week-old GFP-expressing SCID mouse by subcutaneous injection. Implants were harvested after 4 weeks and compared to native murine adipose tissue. Macroscopic view of explant at 4 weeks are depicted in insets. H&E staining of sections taken from the explants at 4 weeks revealed the presence of adipose tissue (adipocytes). Representative H&E pictures are depicted at 10× and 40× are shown in FIG. 9A. Adipocyte area fraction (%), adipocyte density (adipocyte/mm²), and average adipocyte size (um²) in all H&E stained sections were quantified in all groups and compared to native adipose tissue. The groups evaluated were implants containing 1) h-watMSC alone and 2) h-watMSC+h-ECFC.

The data demonstrated that implants containing h-watMSCs+ECFCs presented significantly larger fraction of adipose tissue than those containing h-watMSCs alone after 4 weeks in vivo. This indicated a beneficial effect of high initial microvascular density (FIG. 9) and higher MSC engraftment (FIG. 8) in the presence of ECFC on later adipogenesis. This larger presence of adipose tissue was due to a higher density of adipocytes rather than a difference in the average adipocyte size.

In the Presence of h-ECFC, h-watMSCs Differentiate into Adipocytes after 4 Weeks In Vivo, but h-bmMSC do not

A total of 1.2×10⁶ h-MSCs were resuspended in 200 μL of MATRIGEL™ in the presence of 0.8×10⁶ h-ECFC. h-MSCs were either from human bone marrow (h-bmMSC) or human adipose tissue (h-watMSCs). The mixture was implanted on the back of 6-week-old GFP-expressing SCID mouse by subcutaneous injection (n=4 each group). Implants were harvested after 4 weeks and compared to native murine adipose tissue. Immunofluorescence staining of sections taken from the explants at 4 weeks were carried out with antibodies against gfp and perilipin-A. Explants that used h-watMSC+h-cbECFC or h-bmtMSC+h-cbECFC were analysed and compared with native mouse cutaneous adipose tissue.

The data demonstrated that when h-watMSC are implanted with h-ECFC, the large majority of adipocytes found in the implants at 4 weeks are gfp-negative h-watMSCs that had differentiated into fat cells in vivo (gfp−, perilipin-A+ cells are human adipocyte; data not shown). This indicated that h-watMSCs do differentiate into adipocytes. Host murine adipocytes (gfp+, perilipin-A+ are murine adipocyte; data not shown) could only be seen in the subcutaneous adipose tissue adjacent to the implant. On the other hand, when h-bmMSC are implanted with ECFC, the large majority of adipocytes found in the implants at 4 weeks are murine adipocytes, indicating that h-bmMSC do not differentiate into adipocyte. Collectively, these results indicate that to achieve differentiation of h-MSC into fat in vivo, h-watMSC are preferred over h-bmMSC.

### Example 3

### In Vivo Co-Cultures of ECFCs and Human watMSCs in Collagen/Fibrin-Based Hydrogels

This experiment was performed to show that the same dense microvessel formation and adipogenesis take place in a collagen/fibrin-based gel instead of MATRIGEL™ as shown in Examples 1 and 2 (FIGS. 5 and 9).

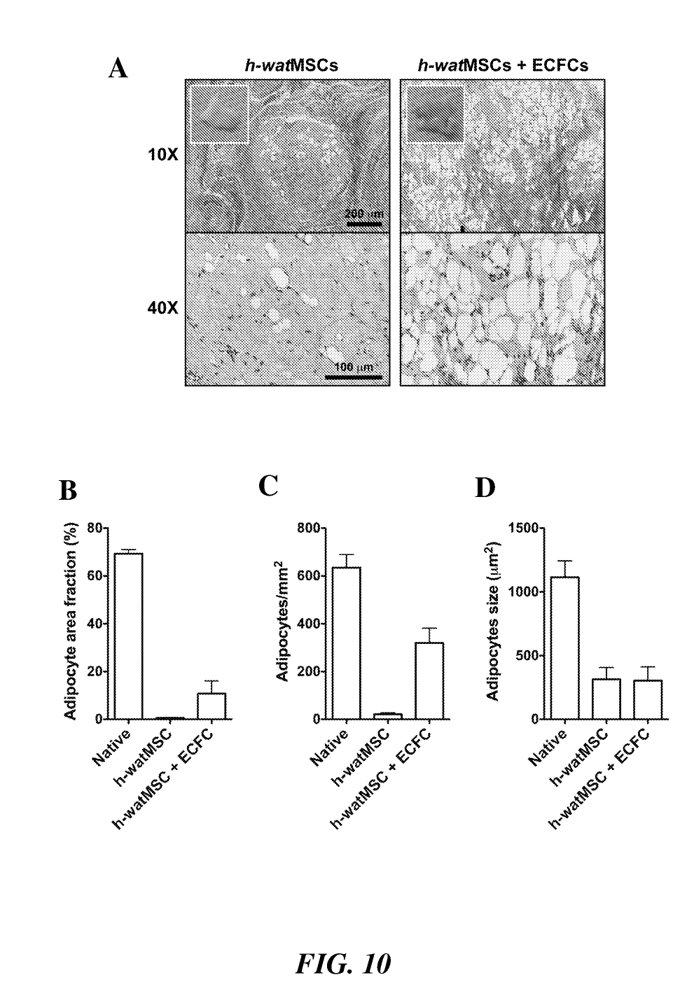

A total of 1.2×10⁶ h-watMSCs were resuspended in 200 μL of a collagen/fibrin-based gel in the presence or absence of 0.8×10⁶ h-ECFC. The mixture was implanted on the back of 6-week-old GFP-expressing SCID mouse by subcutaneous injection. Implants were harvested after 4 weeks and compared to native murine adipose tissue. Macroscopic views of explant at 4 weeks are depicted in insets (FIG. 10A). H&E staining of sections taken from the explants at 4 weeks revealed the presence of adipose tissue (adipocytes). Representative H&E pictures are depicted at 10× and 40×. Adipocyte area fraction (%), adipocyte density (adipocyte/mm²), and average adipocyte size (μm²) were quantified in all groups and compared to native adipose tissue (FIG. 10A). The groups evaluated were implants containing 1) h-watMSC alone and 2) Bh-watMSC+h-ECFC. Bars represent mean±S.D determined from four replicate implants (one mouse each) (FIGS. 10B-10D).

Similar to FIGS. 5 and 9, the inventors showed that collagen/fibrin-based implants containing h-watMSCs and hECFCs presented significantly larger fraction of adipose tissue than those containing h-watMSCs alone after 4 weeks in vivo. As demonstrated previously in Examples 1 and 2, there is a beneficial effect of high initial microvasculardensity (FIG. 8) and higher MSC engraftment (FIG. 7) in the presence of ECFCs on later adipogenesis. This larger presence of adipose tissue was due to a higher density of adipocytes rather than a difference in the average adipocyte size.

### Example 4

### In Vivo Co-Cultures of ECFCs and MSCs in GelMA Hydrogels

This experiment was performed to show that the same dense microvessel formation and adipogenesis take place in GelMA hydrogels instead of MATRIGEL™ and collagen/fibrin-based gels of as shown in Examples 1-3 (FIGS. 5, 9 and 10).

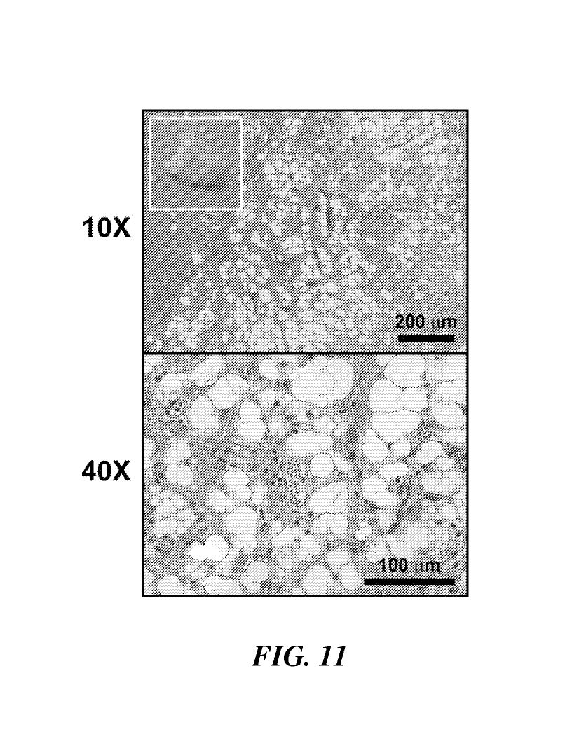

A total of 1.2×10⁶ mouse adipose tissue-derived MSC (m-gfp−watMSCs; isolated from GFP-expressing SCID mice) were resuspended in 200 μL of 1M GelMA (10% w/w gelatin content) in the presence of 0.8×10⁶ h-ECFC. The mixture was injected on the back of 6-week-old nude mouse by subcutaneous injection (n=4 each group). Right after injection, the gel was polymerized by transdermal exposure to 6.9 mW/cm² UV light (360-480 nm; using OmniCure S2000 UV lamp; Lumen Dynamics). Implants were harvested after 4 weeks and compared to native murine adipose tissue. FIG. 11 shows representative H&E stained sections of one implant after 4 weeks in vivo. Macroscopic view shown in the inset. Immunofluorescence staining of sections was also performed. The immunofluorescence staining were carried out with antibodies against gfp and perilipin-A. DAPI was used for nuclear staining.

Similar to FIGS. 5 and 9, FIG. 11 demonstrated that in vivo vascularization and adipogenesis can occur in 1M GelMA. The inventors show that in GelMA implants containing m-gfp−watMSCs and ECFCs presented a large fraction of adipose tissue after 4 weeks in vivo (FIG. 11). The large majority of adipocytes found in the implants at 4 weeks are gfp-positive, indicating the source of the MSCs are donor m-gfp−watMSCs that had differentiated into fat cells in vivo (data not shown). This indicated that watMSC do differentiate into adipocytes. Host murine adipocytes (data not shown) could only be seen in the subcutaneous adipose tissue adjacent to the implant.

In summary, the inventors found that in the presence of EPCs (also known as h-ECFCs) and adipose-derived watMPCs first contribute to vasculogenesis, with the watMPCs serving as perivascular cells and later differentiate to form functional adipocytes. This two-cell-based approach has the potential to improve the engraftment of adipose tissue-derived MPCs and to enhance in vivo adipogenesis.

The references cited herein and throughout the specification are incorporated herein by reference in their entirety.

1. A composition for use in promoting vascularized adipose tissue formation comprising an enriched population of isolated endothelial progenitor cells (EPCs) and an enriched population of isolated mesenchymal progenitor cells (MPCs), wherein the EPCs and MPCs induce the formation of new blood vessels with functional connections to a host vasculature and the formation of new adipose tissue.

2. The composition of claim 1, wherein the vascularized adipose tissue is formed in vitro, in vivo or ex vivo.

3. The composition of claim 1, wherein the new adipose tissue is differentiated from the MPCs.

4. The composition of claim 1, wherein the EPCs are derived from a source selected from a group consisting of bone marrow, cord blood, peripheral blood and blood vessel walls.

5. The composition of claim 1, wherein the MPCs are derived from a source selected from a group consisting of amniotic fluid, bone marrow, cord blood, peripheral blood and adipose tissue.

6. The composition of claim 1, wherein the progenitor cells are autologous to a recipient of the composition.

7. The composition of claim 1, wherein the progenitor cells are allogenic and HLA type matched to a recipient of the composition.

8. (canceled)

9. The composition of claim 1, wherein the composition of enriched populations of EPCs and MPCs further comprising a filler material, wherein the progenitor cells are mixed with the filler material, and the mixture of filler material and progenitor cells is delivered or implanted simultaneously.

10. The composition of claim 9, wherein the filler material is an extracellular matrix.

11. The composition of claim 1, wherein the extracellular matrix has a stiffness property of about 1 Pa to 600 Pa.

12. (canceled)

13. (canceled)

14. (canceled)

15. The composition of claim 1, wherein the enriched population of EPCs is at least 10% but not more than 90% of the cells in the composition.

16. The composition of claim 1, wherein the enriched population of MPCs is at least 10% but not more than 90% of the cells in the composition.

17. The composition of claim 15, wherein the EPCs is at least 40% of the cells in the composition.

18. The composition of claim 1, wherein the composition further comprise pre-differentiated MPCs.

19. The composition of claim 18, wherein the MPCs have been pre-differentiated in vitro.

20. The composition of claim 18, wherein the MPCs have been pre-differentiated to pre-adipocytes and/or adipocytes.

21. The composition of claim 18, wherein the new adipose tissue is from the pre-differentiated MPCs.

22. A method of promoting vascularized adipose tissue formation in a subject in need thereof comprising implanting a composition comprising an enriched population of isolated endothelial progenitor cells (EPCs) and an enriched population of isolated mesenchymal progenitor cells (MPCs), wherein the EPCs and MPCs induce the formation of new blood vessels with functional connections to the subject's vasculature and the formation of new adipose tissue.

23. (canceled)

24. (canceled)

25. (canceled)

26. (canceled)

27. (canceled)

28. The method of claim 22, wherein the composition of enriched populations of EPCs and MPCs are mixed with a filler material and the progenitor cells are delivered simultaneously.

29. The method of claim 28, wherein the filler material is an extracellular matrix.

30. (canceled)

31. (canceled)

32. (canceled)

33. (canceled)

34. (canceled)

35. (canceled)

36. The method of claim 22, wherein the composition further comprise pre-differentiated MPCs, wherein the MPCs have been pre-differentiated in vitro.

37. The method of claim 36, wherein the MPCs have been pre-differentiated to pre-adipocytes or adipocytes.

38. A composition for promoting vascularized adipose tissue formation comprising:
a. an enriched population of isolated endothelial progenitor cells (EPCs);
b. an enriched population of isolated mesenchymal progenitor cells (MPCs);
c. an enriched population of pre-differentiated MPCs and;
d. a pharmaceutically acceptable carrier.

39. The composition of claim 38, wherein the MPCs have been pre-differentiated to pre-adipocytes or adipocytes.

40. (canceled)

41. (canceled)

42. (canceled)

43. (canceled)

44. (canceled)

45. (canceled)

46. (canceled)

47. The composition of claim 38, further comprising a filler material.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)
